(19) United States
(12) Patent Application Publication
(10) Pub. No.: US 2006/0142203 A1
(43) Pub. Date: Jun. 29, 2006

(54) Compositions and methods comprising protein activated receptor antagonists

(76) Inventors: Todd Hembrough, Damascus, MD (US); Victor Pribluda, Silver Spring, MD (US)

Correspondence Address: JOHN S. PRATT, ESQ;KILPATRICK STOCKTON, LLP, 1100 PEACHTREE STREET, ATLANTA, GA, 30309, US

(21) Appl. No.: 10/608,886

(22) Filed: Jun. 26, 2003

Related U.S. Application Data
(60) Provisional application No. 60/391,655, filed on Jun. 26, 2002.
(60) Provisional application No. 60/398,662, filed on Jul. 26, 2002.
(60) Provisional application No. 60/458,095, filed on Mar. 27, 2003.
(60) Provisional application No. 60/466,296, filed on Apr. 29, 2003.

(51) Int. Cl. A61K 38/08; A61K 38/06; A61K 38/05; A61K 38/04

(52) U.S. Cl. 514/17; 514/18; 514/19

(57) ABSTRACT

Compositions and methods comprising protein activated receptor antagonists are provided More particularly, the present invention relates to the use of proteins, peptides and biomolecules that bind to protein activated receptor 2, and inhibit the processes associated with the activation of that receptor. More specifically, the present invention provides novel compositions and methods for the treatment of disorders and diseases such as those associated with abnormal cellular proliferation, angiogenesis, inflammation and cancer.

### CROSS REFERENCE TO RELATED APPLICATIONS

The present application claims priority to U.S. Provisional Application Ser. No. 60/391,655 filed Jun. 26, 2002, U.S. Provisional Application Ser. No. 60/398,662 filed Jul. 26, 2002, U.S. Provisional Application Ser. No. 60/458,095 filed Mar. 27, 2003 and U.S. Provisional Application Ser. No. 60/466,296 filed Apr. 29, 2003.

### FIELD OF THE INVENTION

The present invention relates to compositions and methods comprising protein activated receptor antagonists. More particularly, the present invention relates to the use of proteins, peptides and biomolecules that bind to protein activated receptors, and inhibit the processes associated with the activation of that receptor. More specifically, the present invention provides novel compositions and methods for the treatment of disorders and diseases such as those associated with abnormal cellular proliferation, angiogenesis, inflammation and cancer.

### BACKGROUND OF THE INVENTION

Cellular proliferation is a normal ongoing process in all living organisms and is one that involves numerous factors and signals that are delicately balanced to maintain regular cellular cycles. The general process of cell division is one that consists of two sequential processes: nuclear division (mitosis), and cytoplasmic division (cytokinesis). Because organisms are continually growing and replacing cells, cellular proliferation is a central process that is vital to the normal functioning of almost all biological processes. Whether or not mammalian cells will grow and divide is determined by a variety of feedback control mechanisms, which include the availability of space in which a cell can grow, and the secretion of specific stimulatory and inhibitory factors in the immediate environment.

When normal cellular proliferation is disturbed or somehow disrupted, the results can affect an array of biological functions. Disruption of proliferation could be due to a myriad of factors such as the absence or overabundance of various signaling chemicals or presence of altered environments. Some disorders characterized by abnormal cellular proliferation include cancer, abnormal development of embryos, improper formation of the corpus luteum, difficulty in wound healing as well as malfunctioning of inflammatory and immune responses.

Cancer is characterized by abnormal cellular proliferation. Cancer cells exhibit a number of properties that make them dangerous to the host, often including an ability to invade other tissues and to induce capillary ingrowth, which assures that the proliferating cancer cells have an adequate supply of blood. One of the defining features of cancer cells is that they respond abnormally to control mechanisms that regulate the division of normal cells and continue to divide in a relatively uncontrolled fashion until they kill the host.

Angiogenesis and angiogenesis related diseases are closely affected by cellular proliferation. As used herein, the term “angiogenesis” means the generation of new blood vessels into a tissue or organ. Under normal physiological conditions, humans or animals undergo angiogenesis only in very specific restricted situations. For example, angiogenesis is normally observed in wound healing, fetal and embryonal development and formation of the corpus luteum, endometrium and placenta. The term “endothelium” is defined herein as a thin layer of flat cells that lines serous cavities, lymph vessels, and blood vessels. These cells are defined herein as “endothelial cells”. The term “endothelial inhibiting activity” means the capability of a molecule to inhibit angiogenesis in general. The inhibition of endothelial cell proliferation also results in an inhibition of angiogenesis.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

Persistent, unregulated angiogenesis occurs in a multiplicity of disease states, tumor metastasis and abnormal growth by endothelial cells and supports the pathological damage seen in these conditions. The diverse pathological disease states in which unregulated angiogenesis is present have been grouped together as angiogenic-dependent, angiogenic-associated, or angiogenic-related diseases. These diseases are a result of abnormal or undesirable cell proliferation, particularly endothelial cell proliferation.

The hypothesis that tumor growth is angiogenesis-dependent was first proposed in 1971 by Judah Folkman (N. Engl. Jour. Med. 285:1182 1186, 1971). In its simplest terms the hypothesis proposes that once tumor “take” has occurred, every increase in tumor cell population must be preceded by an increase in new capillaries converging on the tumor. Tumor “take” is currently understood to indicate a prevascular phase of tumor growth in which a population of tumor cells occupying a few cubic millimeters volume and not exceeding a few million cells, survives on existing host microvessels. Expansion of tumor volume beyond this phase requires the induction of new capillary blood vessels. For example, pulmonary micrometastases in the early prevascular phase in mice would be undetectable except by high power microscopy on histological sections. Further indirect evidence supporting the concept that tumor growth is angiogenesis dependent is found in U.S. Pat. Nos. 5,639,725, 5,629,327, 5,792,845, 5,733,876, and 5,854,205, all of which are incorporated herein by reference.

Thus, it is clear that cellular proliferation, particularly endothelial cell proliferation, and most particularly angiogenesis, plays a major role in the metastasis of a cancer. If this abnormal or undesirable proliferation activity could be repressed, inhibited, or eliminated, then the tumor, although present, would not grow. In the disease state, prevention of abnormal or undesirable cellular proliferation and angiogenesis could avert the damage caused by the invasion of the new microvascular system. Therapies directed at control of the cellular proliferative processes could lead to the abrogation or mitigation of these diseases.

Recently studies have been conducted that correlate abnormal protein activated receptor activity with certain disorders and diseases. Of particular interest is protein activated receptor-2 which has been discovered to be associated with disorders such as inflammation, angiogenesis, and sepsis. Although several attempts have been made, no effective antagonists of protein activated receptor-2 have been identified.

What is needed are compositions and methods that can inhibit abnormal or undesirable cellular function, especially functions that are associated with undesirable cellular proliferation, angiogenesis, inflammation and cancer. The compositions should comprise proteins, peptides and biomolecules that overcome the activity of endogenous protein activated receptor ligands and prevent the activation of protein activated receptors thereby inhibiting the development of abnormal physiological states associated with inappropriate protein activated receptor activation. Finally, the compositions and methods for inhibiting protein activated receptor activation should preferably be non-toxic and produce few side effects.

### SUMMARY OF THE INVENTION

Compositions and methods are provided that are effective in inhibiting abnormal or undesirable cell function, particularly cellular activity and proliferation related to angiogenesis, neovascularization, inflammation, tumor growth, sepsis, neurogenic and inflammatory pain, asthma and post operative ileus. The compositions comprise a naturally occurring or synthetic protein, peptide, protein fragment or biomolecule containing all, or an active portion of a ligand that binds protein activated receptors, optionally combined with a pharmaceutically acceptable carrier.

Representative ligands or antagonists useful for the present invention comprise proteins, peptides and biomolecules that bind protein activated receptors, such as, but not limited to, protein activated receptor 1 (PAR-1) or protein activated receptor 2 (PAR-2), protein activated receptor 3 (PAR-3), and protein activated receptor 4 (PAR-4). Preferred ligand compositions of the present invention, include but are not limited to, proteins comprising LIGK (SEQ ID NO:1), LIGKV (SEQ ID NO:2), KGIL (SEQ ID NO:3), KGI (SEQ ID NO:4), AGI (SEQ ID NO:5), IGA (SEQ ID NO:6), KGA (SEQ ID NO:7), KGA (SEQ ID NO:8), KAI (SEQ ID NO:9), IAK (SEQ ID NO:10), RGI (SEQ ID NO:11), IGR (SEQ ID NO:12), Dab-GI (Dab=diamino butanoic acid) (SEQ ID NO:13 ), Dap-GI (Dap=diamino proprionic acid) (SEQ ID NO:14), IG-Dab (SEQ ID NO:15), IG-Dap (SEQ ID NO:16), LIG-Dab (SEQ ID NO:17), Dab-GIL (SEQ ID NO:18), LIG-Dap (SEQ ID NO:19), Dap-GIL (SEQ ID NO:20), LIG-Orn (SEQ ID NO:21), Orn-GIL (SEQ ID NO:22), Orn-GI (SEQ ID NO:23) and IG-Orn (SEQ ID NO:24), ENMD 545 (FIG. 1), ENMD 547 (FIG. 1), and various peptidomimetic structures provided in FIG. 2. Also contemplated within the scope of this invention are ligands and antagonists that comprise functional and structural derivatives and equivalents of the above-listed biomolecules.

Preferably, the protein, peptide, protein fragment or biomolecule contains all or an active portion of the above identified ligands and antagonists. The term “active portion”, as used herein, means a portion of a protein, peptide or biomolecule that inhibits protein activated receptor activation. Also included in the present invention are homologs, peptides, or protein fragments, or combinations thereof of the above-identified ligands and antagonists, that inhibit protein activated receptor activity.

It is believed that by inhibiting protein activated receptor activity, the methods and compositions described herein are useful for inhibiting diseases and disorders associated with abnormal protein activated receptor activity. The methods provided herein for treating diseases and processes mediated by protein activated receptors, such as inflammation and cancer, involve administering to a human or animal the composition described herein in a dosage sufficient to inhibit protein activated receptor activity, particularly PAR-2 activity. The methods are especially useful for treating or repressing the growth of tumors, particularly by inhibiting angiogenesis.

Accordingly, it is an object of the present invention to provide methods and compositions for treating diseases and processes that are mediated by abnormal or undesirable protein activated receptor activity.

Another object of the present invention is to provide methods and compositions for inhibiting abnormal or undesirable cell function, particularly cellular activity and proliferation related to angiogenesis, neovascularization, inflammation, tumor growth, sepsis, neurogenic and inflammatory pain, asthma and post operative ileus.

It is another object of the present invention to provide methods and compositions for treating or repressing the growth of a cancer.

It is yet another object of the present invention to provide methods and compositions for therapy of cancer that has minimal side effects.

It is another object of the present invention to provide methods and compositions for treating diseases and processes that are mediated by angiogenesis.

Yet another object of the present invention is to provide methods and compositions comprising the use of proteins, peptides, biomolecules, active fragments and homologs thereof that inhibit protein activated receptor activity.

Another object of the present invention is to provide methods and compositions for treating diseases and processes that are mediated by angiogenesis by administrating antiangiogenic compounds comprising ligands that bind protein activated receptor activity.

It is a further object of the present invention to provide methods and compositions for treating diseases and processes that are mediated by abnormal protein activated receptor activity.

It is another object of the present invention to provide methods and compositions for diagnosing diseases and disorders by measuring abnormal protein activated receptor activity.

It is still another object of the present invention to provide compositions comprising ligands that bind protein activated receptors wherein the compositions further comprise pharmaceutically acceptable carriers.

Yet another object of the present invention is to provide methods and compositions comprising ligands that bind protein activated receptors wherein the compositions further comprise pharmaceutically acceptable carriers that may be administered intramuscularly, intravenously, transdermally, orally, or subcutaneously.

It is yet another object of the present invention to provide compositions and methods for treating diseases and processes that are mediated by angiogenesis including, but not limited to, hemangioma, solid tumors, blood borne tumors, leukemia, metastasis, telangiectasia, psoriasis, scleroderma, pyogenic granuloma, myocardial angiogenesis, Crohn's disease, plaque neovascularization, arteriovenous malformations, corneal diseases, rubeosis, neovascular glaucoma, diabetic retinopathy, retrolental fibroplasia, arthritis, diabetic neovascularization, macular degeneration, wound healing, peptic ulcer, Helicobacter related diseases, fractures, keloids, vasculogenesis, hematopoiesis, ovulation, menstruation, placentation, and cat scratch fever.

These and other objects, features and advantages of the present invention will become apparent after a review of the following detailed description of the disclosed embodiment and the appended claims.

### BRIEF DESCRIPTION OF THE FIGURES

FIG. 1 provides schematics showing the structures of ENMD 547 and ENMD 545.

FIG. 2A-2CC provides a list of peptidomimetic structures comprising PAR-2 antagonists.

FIG. 3 provides a schematic showing a proposed interaction of an antagonist with PAR-2.

FIG. 4A shows calcium mobilization curves of the PAR-2 agonist SLIGKV (SEQ ID NO:25) compared with two truncated molecules LIGK (SEQ ID NO:1) and LIGKV (SEQ ID NO:2). FIG. 4B shows the results of an in vitro assay demonstrating PAR-2 signaling in response to PAR-2 activating peptide and its alanine-substituted analogs. FIG. 4C shows the results of an in vitro assay demonstrating PAR-2 signaling in response to AP2 and its truncated forms and alanine substituted analogs.

FIG. 5 shows a representative dosing study where increasing concentrations of LIGK (SEQ ID NO:1) were used to block P2AP signaling.

FIG. 6 provides a graph showing the results of an in vitro inhibition study in the presence of LIGK (SEQ ID NO:1) or LIGKV (SEQ ID NO:2).

FIG. 7 provides a graph showing the effect of LIGK (SEQ ID NO:1) on PAR-2 signaling.

FIG. 8 provides the effect of LIGK (SEQ ID NO:1) on the PAR-2 edema model.

FIG. 9 provides a graph showing the inhibitory effect of LIGK (SEQ ID NO:1).

FIG. 10 provides a graph showing the inhibitory effect of LIGK (SEQ ID NO:1) on metatstatic tumor growth.

FIG. 11 provides a graph demonstrating dose dependency across multiple independent studies, with an approximate IC50 of 2 mg/day.

FIG. 12 shows inhibition of LLC primary tumor growth by LIGK (SEQ ID NO:1).

FIG. 13 shows the results of a matrigel angiogenesis assay demonstrating the inhibitory effect of LIGK (SEQ ID NO:1).

FIG. 14 provides a graph showing a decrease in AP2 stimulated signaling in the presence of ENMD 547.

FIG. 15 shows the effect of ENMD 547 on ATP and AP2 signaling.

FIG. 16 shows the results of an inhibition study comparing the effects of LIGK (SEQ ID NO:1) versus ENMD 547 on metastatic tumor growth.

FIG. 17 provides a flow chart showing the peptidomimetic approach taken by the inventors.

FIG. 18 provides a schematic showing peptidomimetic design.

FIG. 19 provides the results of an inflammation (arthritis) study conducted to demonstrate the effect of LIGK (SEQ ID NO:1) on mice.

FIG. 20 shows attenuation of arthritis in mice in the presence of LIGK (SEQ ID NO:1) (referred to as ENMD 520).

FIG. 21 shows attenuation of arthritis in the presence of LIGK (SEQ ID NO:1) ENMD 520 and ENMD 547.

FIG. 22 shows prevention of weight loss in the presence of LIGK (SEQ ID NO:1), ENMD 520.

FIG. 23 provides antitumor data for LIGK (SEQ ID NO:1) and ENMD 547.

FIG. 24 provides addition peptidomimetic structures for PAR-2 antagonists.

FIG. 25 provides results of an inhibition study using fragments, scrambled and reverse peptides.

### DETAILED DESCRIPTION

The following description includes the best presently contemplated mode of carrying out the invention. This description is made for the purpose of illustrating the general principles of the inventions and should not be taken in a limiting sense. The entire text of the references mentioned herein are hereby incorporated in their entireties by reference, including U.S. Provisional Application Ser. No. 60/391,655 filed Jun. 26, 2002, U.S. Provisional Application Ser. No. 60/398,662 filed Jul. 26, 2002, U.S. Provisional Application Ser. No. 60/458,095 filed Mar. 27, 2003 and U.S. Provisional Application Ser. No. 60/466,296 filed Apr. 29, 2003.

Proteinase activated receptor-2 (PAR-2) is a seven transmembrane G-protein coupled receptor (GPCR) which signals in response to the proteolytic activity of trypsin, tryptase, matriptase, the tissue factor (TF)/factor VIIa (fVIIa) complex and other proteases such as neutrophil protease-3. Proteolytic cleavage of the amino terminus results in the unveiling of a new amino terminus that activates the receptor through a tethered peptide ligand mechanism; essentially the terminus becomes the ligand which inserts into the ligand binding pocket of the receptor. The short synthetic activating peptide (PAR 2AP, SLIGKV (SEQ ID NO:25) (human), SLIGRL-NH₂ (mouse) (SEQ ID NO:26)) activates the receptor. Upon binding of the ligand, there is an increase in intracellular calcium concentration.

Several studies have demonstrated that PAR-2 is involved in angiogenesis, neovascularization and inflammation. PAR-2 has also been associated with pain transmission, tissue injury and regulation of cardiovascular function. For example, Milia et al. discuss the wide expression of PAR-2 in the cardiovascular system, mediation of endothelial cell mitogenesis in vitro by PAR-2, and promotion of vasodilation and microvascular permeability in vivo by PAR-2: all of these steps are regarded as essential steps in angiogenesis. (Milia et al. Circulation Research Vol.91 (4) 2002 pp.346-352) Milia et al. further discuss upregulation of PAR-2 expression by cytokines, including tumor necrosis factor-α, interleukin-β, and lipopolysaccharide, all thought to be involved in inflammation. (Id.)

In addition, recent studies have shown that PAR-2 activation mediates neurogenic inflammation and nociception, illustrating that in some cases, activation of PAR-2 on neurons leads to the generation of proinflammatory cytokines, and a panoply of inflammatory signals. PAR-2 has also been shown to play an essential role in the onset of chronic inflammatory diseases such as rheumatoid arthritis.

Based on the current knowledge of PAR-2 activity in abnormal physiological states, it is believed that PAR-2 activity is associated with numerous disorders and diseases, including but not limited to angiogenesis, neovascularization, inflammation, tumor growth, sepsis, neurogenic and inflammatory pain, asthma and post operative ileus.

The present inventors have shown herein that the proteolytic activity of the PAR-2 agonist TF/fVIIa promotes tumor growth and angiogenesis independently of its role in coagulation. Further characterization and analysis of the role of PAR-2 and its involvement in disease has been difficult, because until now, no specific antagonists of PAR-2 had been identified. Here the inventors describe for the first time specific antagonists of PAR-2 signaling. In vivo, these PAR-2 antagonists are potent inhibitors of angiogenesis and tumor growth. Since previous studies by the inventors suggested a possible role for PAR-2 in tumor growth and angiogenesis, these inhibitors were further assessed to determine if they could inhibit tumor growth or angiogenesis. In vivo, inhibition of PAR-2 signaling results in potent inhibition of both angiogenesis and tumor growth. Thus, these inhibitor studies demonstrate that PAR-2 activity regulates angiogenesis and tumor growth. These data support the inventors' finding of potent and specific antagonists of PAR-2 signaling which promise to be a powerful tools for the study of PAR-2 physiology in normal and pathological processes.

The studies described herein provide the first identification of PAR-2 antagonists. Numerous reports have been published demonstrating important physiological functions of PAR-2. These activities range from nociception, to inflammation, asthma, and neurogenic pain. In each of these studies specific mention is made to the absence of specific PAR-2 antagonist and their great value in the future characterization of this receptor.

Despite the acknowledgement by the scientific and medical community for PAR-2 antagonists based on the discovery that PAR-2 is associated with several diseases and disorders, the long felt need for such antagonists had not been satisfied until the present discovery. Indeed although other studies claim to describe methods that involve inhibiting PAR-2 activity, none of them actually identify specific antagonists, for example, one such study focuses instead on blocking proteolytic cleavage of the PAR-2 amino terminal by trypsin, tryptase, matriptase or the tissue factor (TF)/factor VIIa (fVIIa) complex (see for example WO 01/52883 A1). Such studies acknowledge the need for PAR-2 antagonists, but fail to define any specific peptides or provide any guidance with regard to potentially successful conformations or configurations for such peptides, proteins or biomolecules. The present inventors however have overcome these failures and have successfully identified specific peptides as well as discovered certain conformations of protein/peptide structures that enable the design and elucidation of PAR-2 antagonists.

As discussed above, PAR's are a family of G-protein coupled receptors that function as sensors of thrombotic or inflammatory proteinase activity. Knockout mice lacking the PAR-2 receptor demonstrated little joint swelling or tissue damage in an adjuvant monoarthritis model of chronic inflammation, thereby re-confirming the role of PAR-2 in inflammation. In another experiment, the inventors showed that the tissue factor coagulation pathway was required for the growth of both primary and metastatic tumors. This required the activity of TF/fVIIa complex, but not fXa, which is the normal, physiological target of TF/fVIIa activity. Accordingly, though not wishing to be bound by the following theory, it is believed that in abnormal physiological states, the TF/fVIIa complex is targeting something other than fXa, and based on the studies herein, the inventors believe that the target is PAR-2.

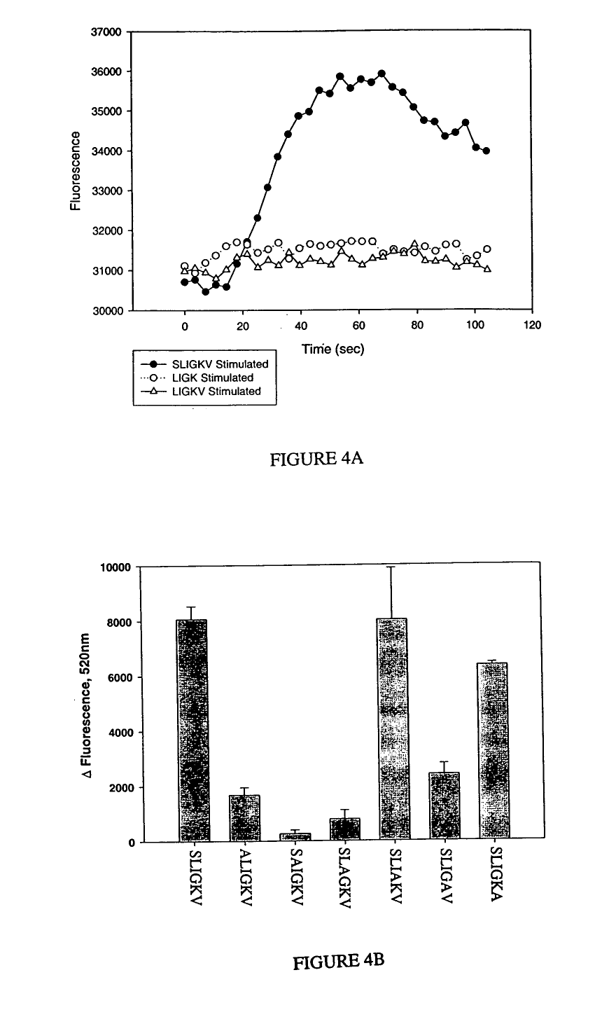

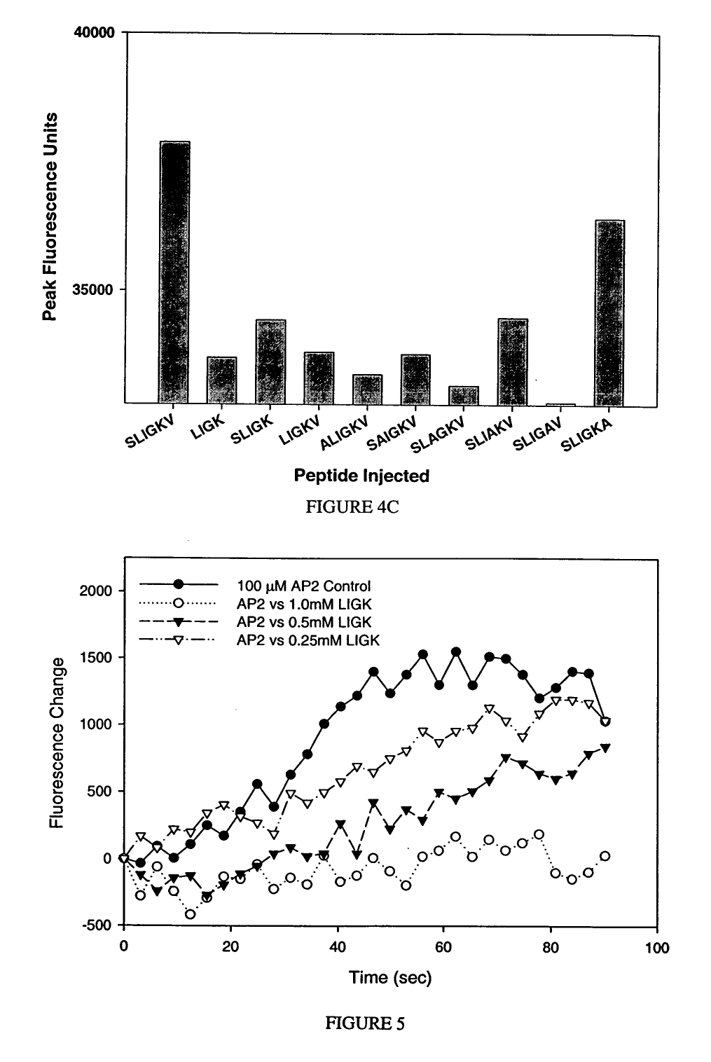

In order to design a peptide antagonist for PAR-2, the inventors first mapped the signaling activity of the agonist peptide, SLIGKV (SEQ ID NO:25) (this signaling peptide is also known as P2AP or 2AP or AP2 in the scientific literature) which was either truncated or monosubstituted with alanine. This was done in order to exclude those peptides that retained signaling activity, and would desensitize cells in inhibition studies. FIG. 4A shows calcium mobilization curves of the PAR-2 agonist SLIGKV (SEQ ID NO:25) compared with two truncated molecules LIGK (SEQ ID NO:1) and LIGKV (SEQ ID NO:2). Neither truncated molecule was able to induce calcium mobilization, in contrast with SLIGKV (SEQ ID NO:25), which demonstrates the typical spike of calcium release followed by degradation of signal. Similar studies were performed on alanine substituted SLIGKV (SEQ ID NO:25) peptides (FIGS. 4B and 4C). It was found that substitution of SLIGKV at S, L, I, or K abrogated or significantly diminished signaling activity, while two substituted peptides, SLIAKV (SEQ ID NO:31) and SLIGKA (SEQ ID NO:33) demonstrated robust signaling activity.

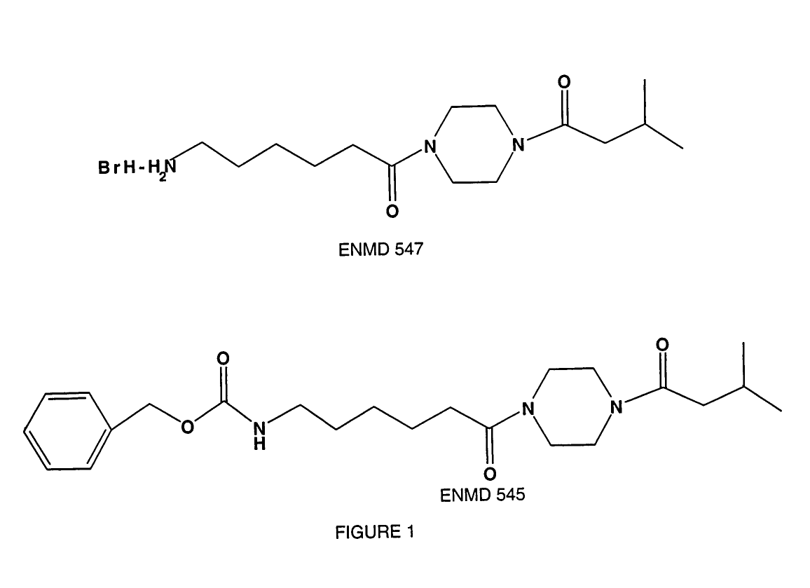

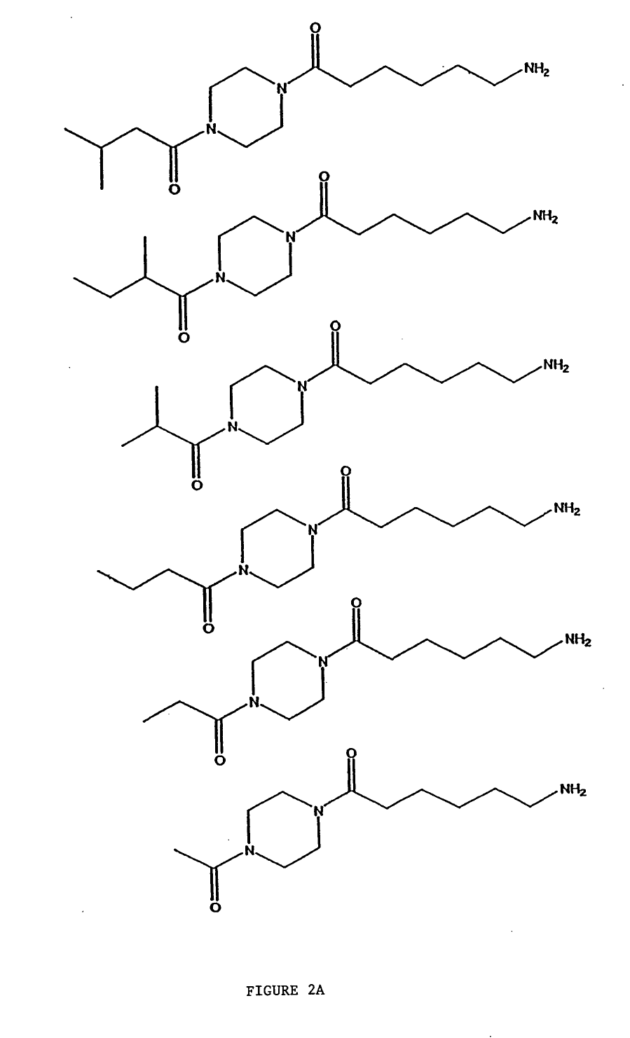

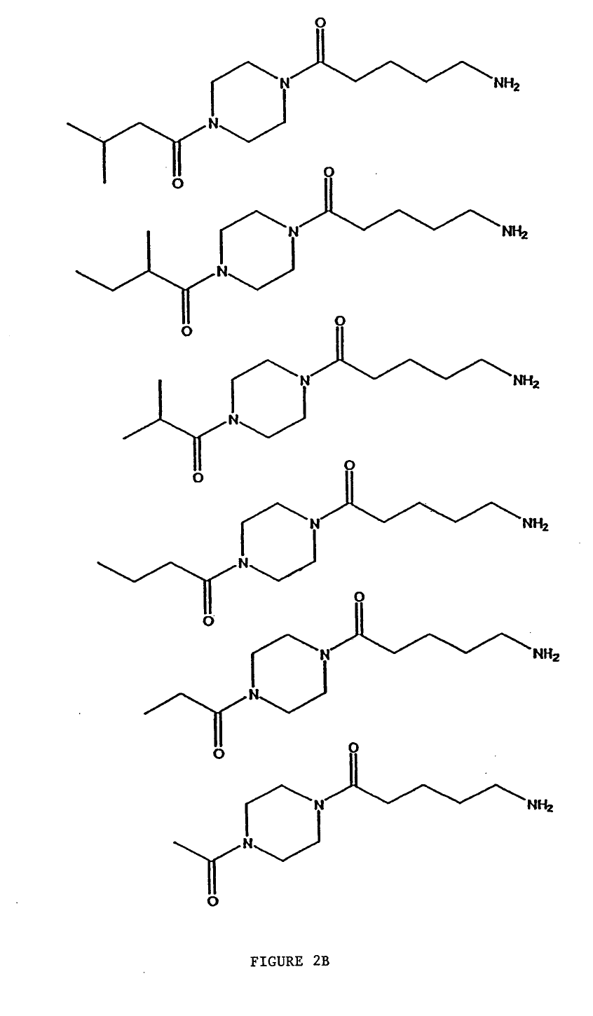

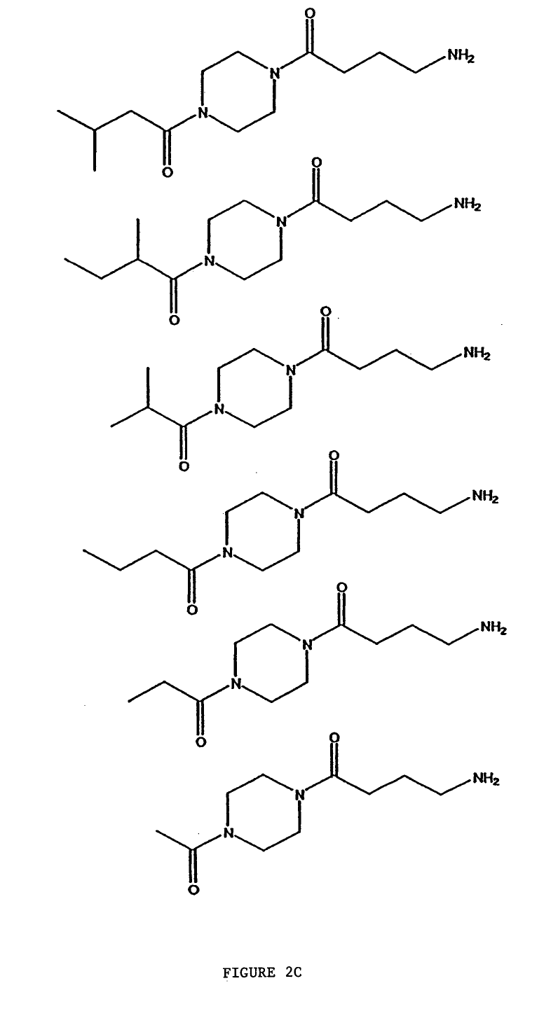

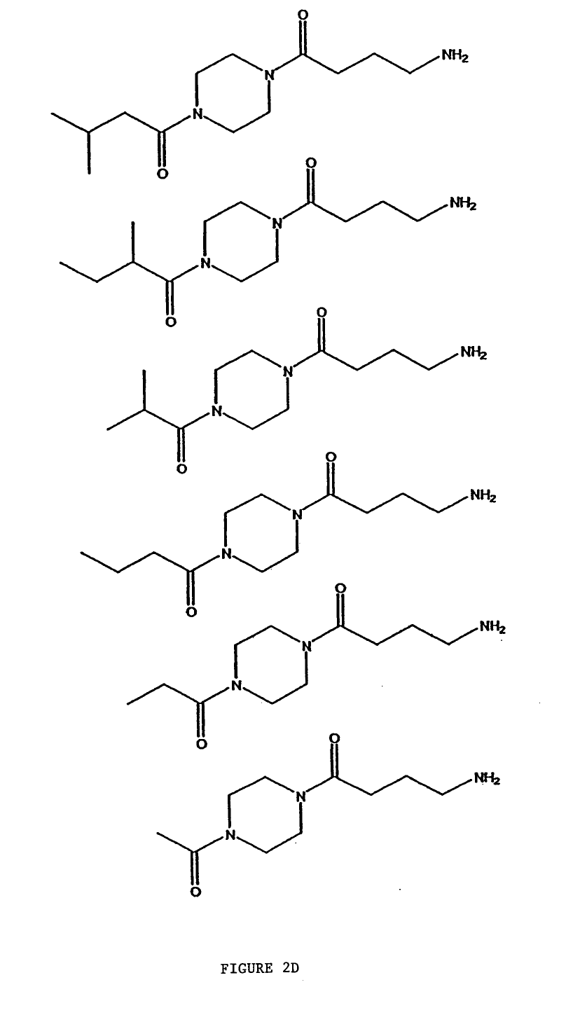

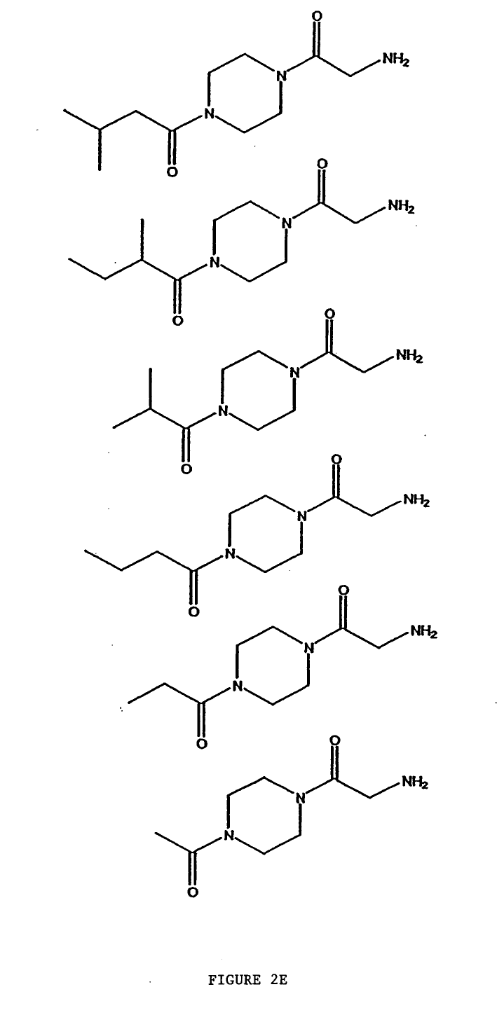

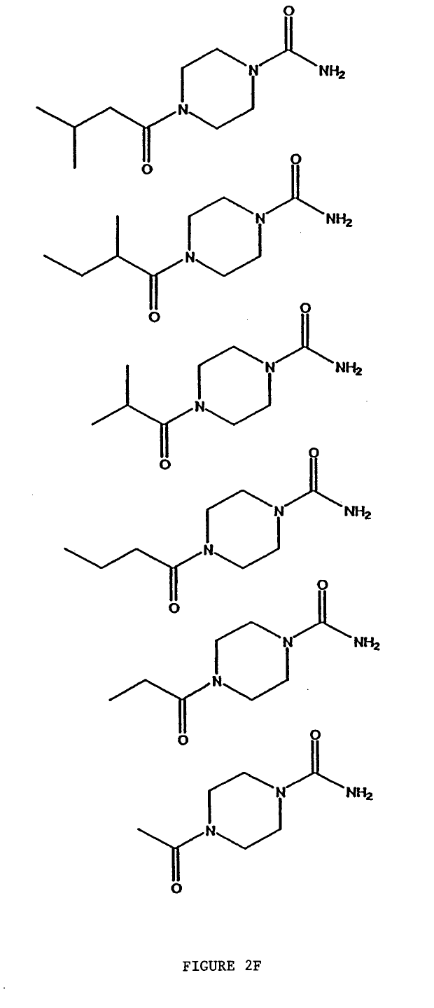

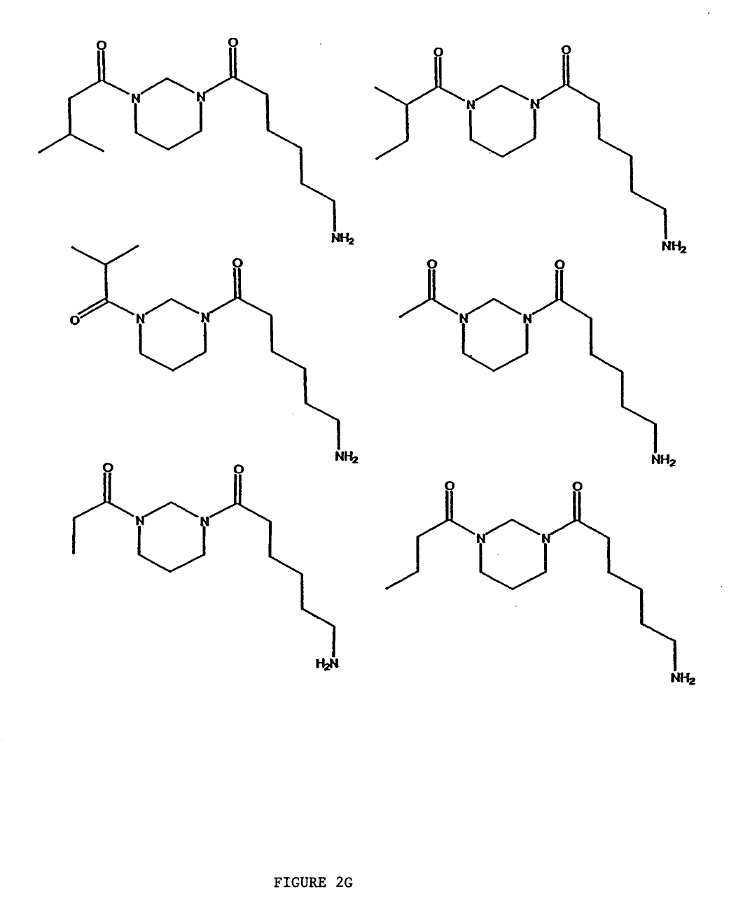

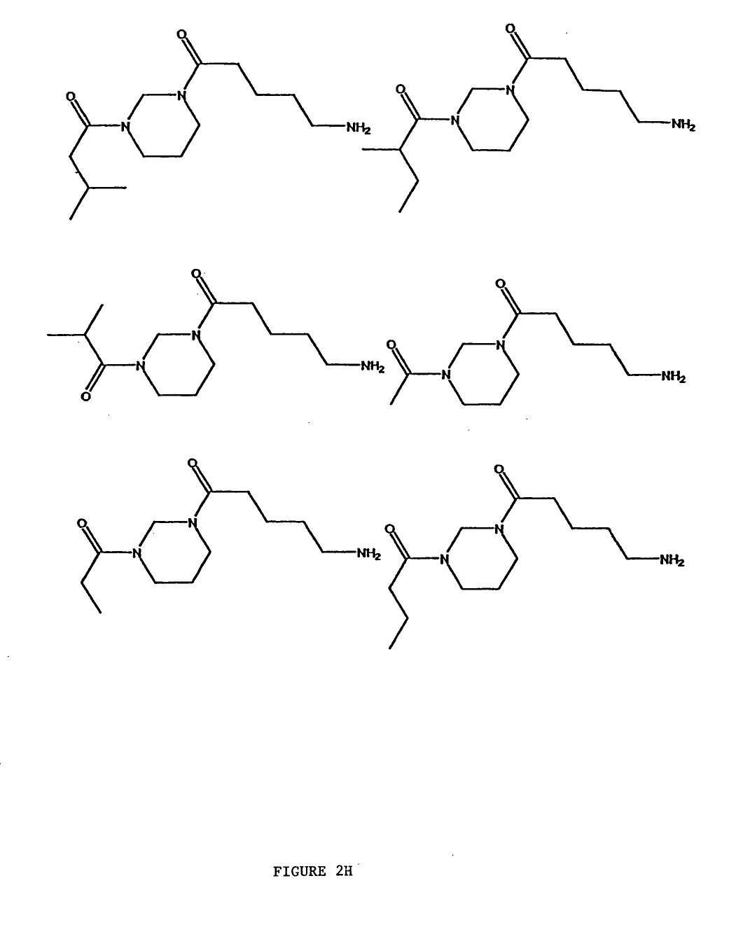

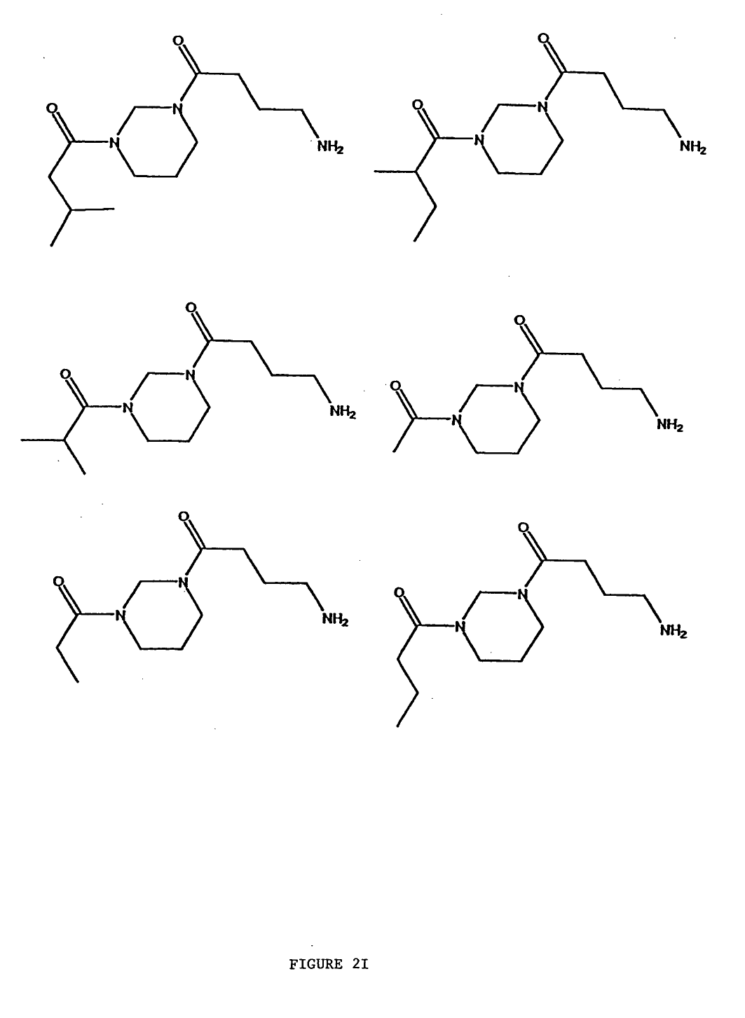

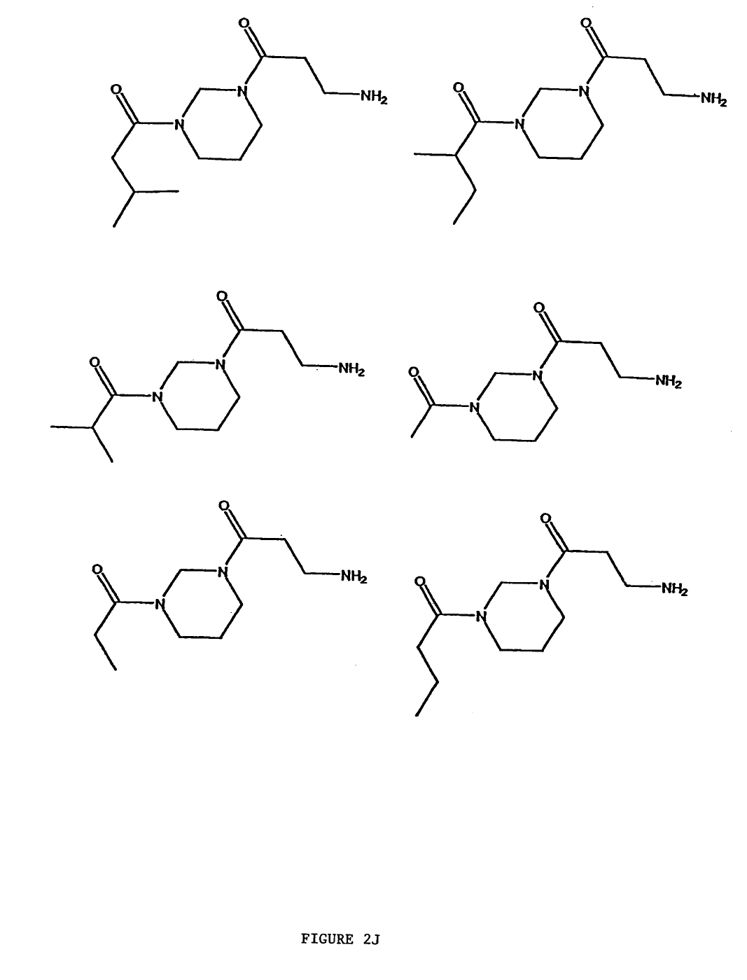

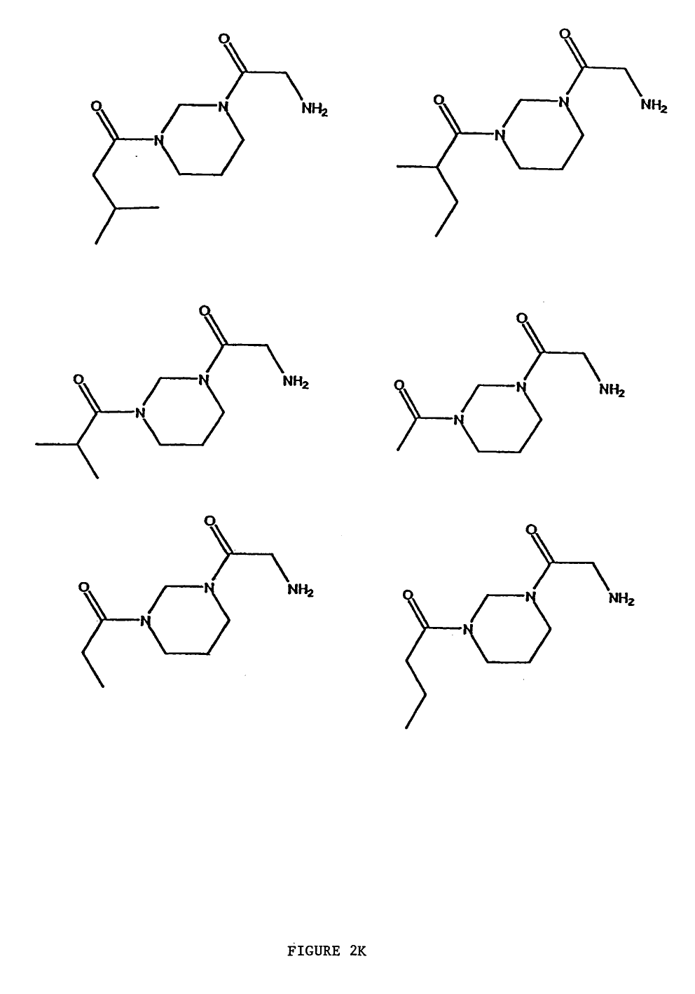

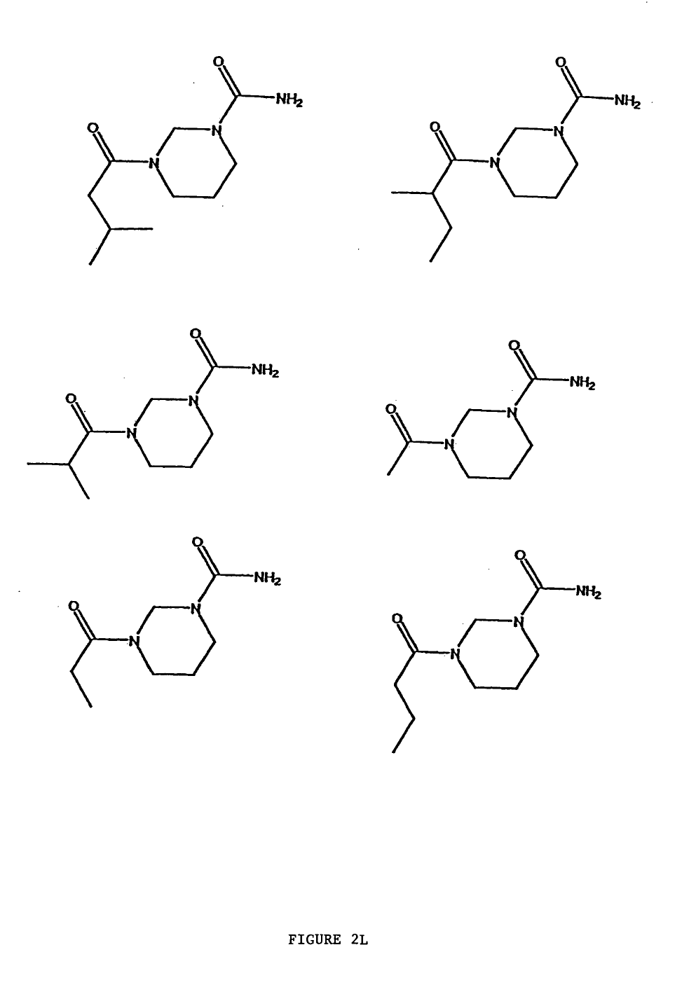

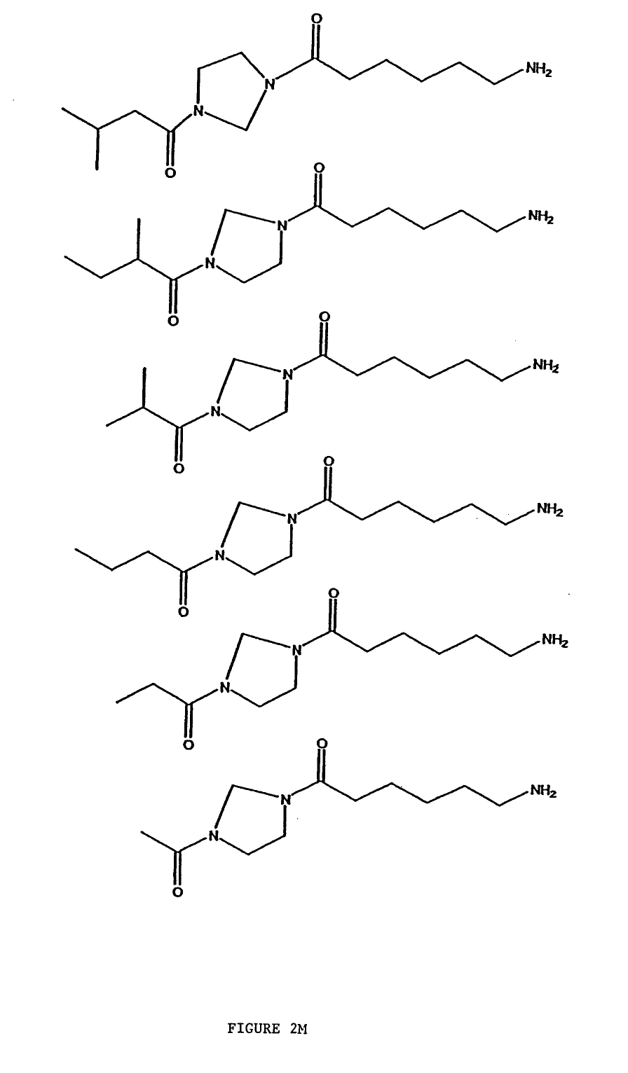

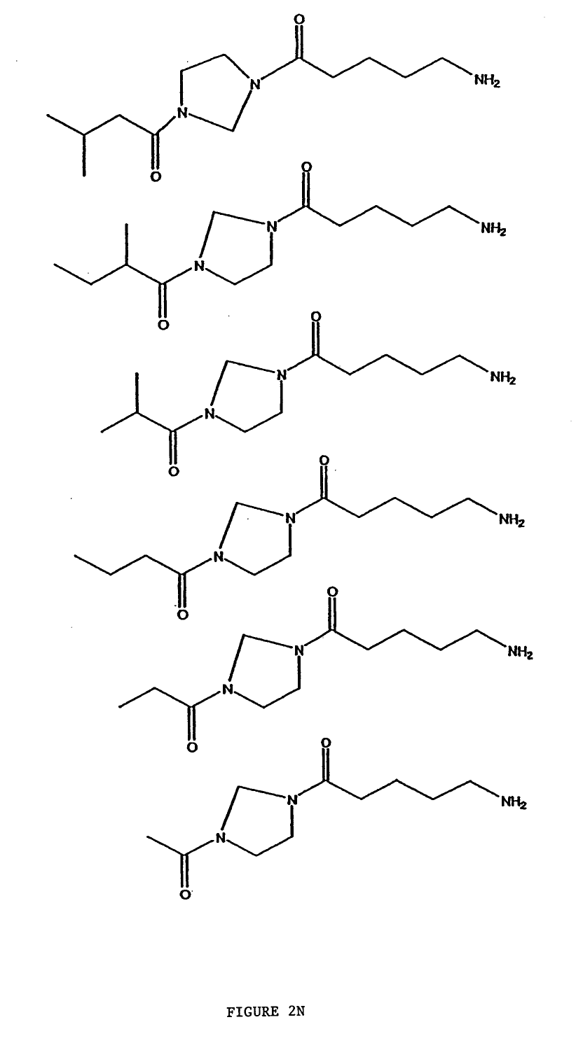

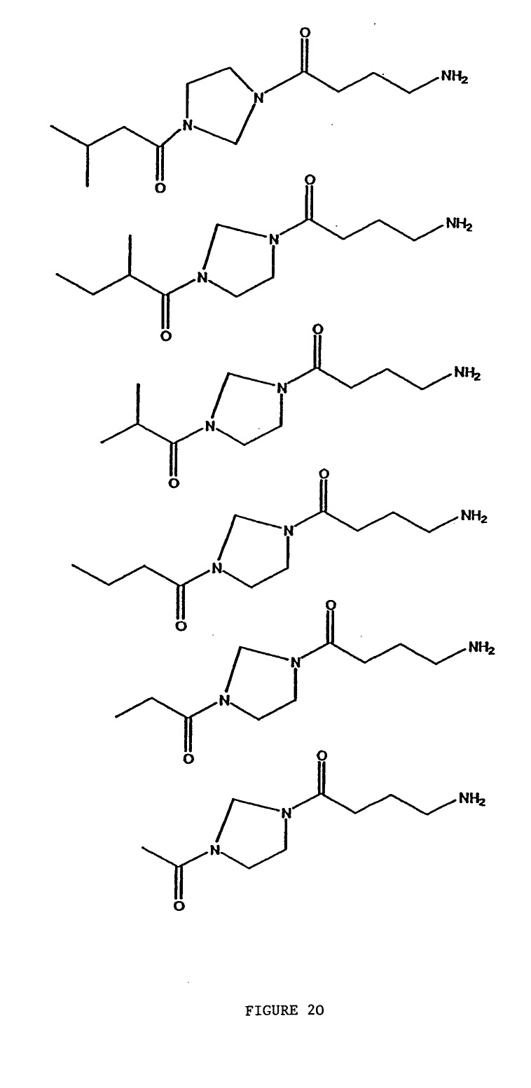

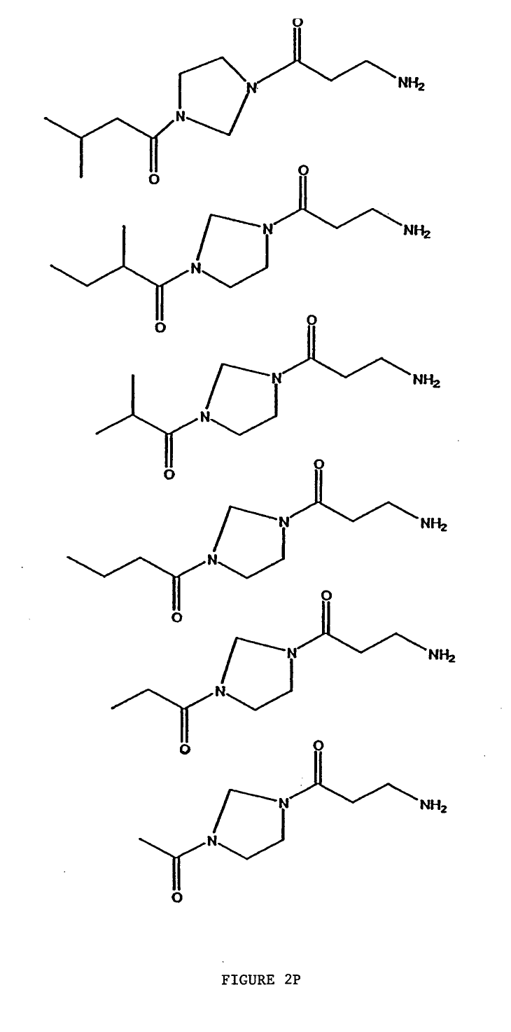

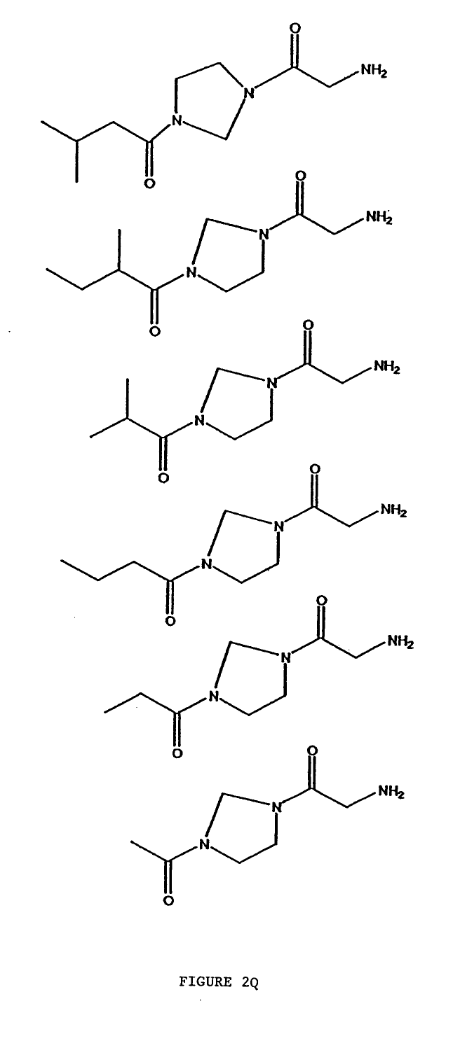

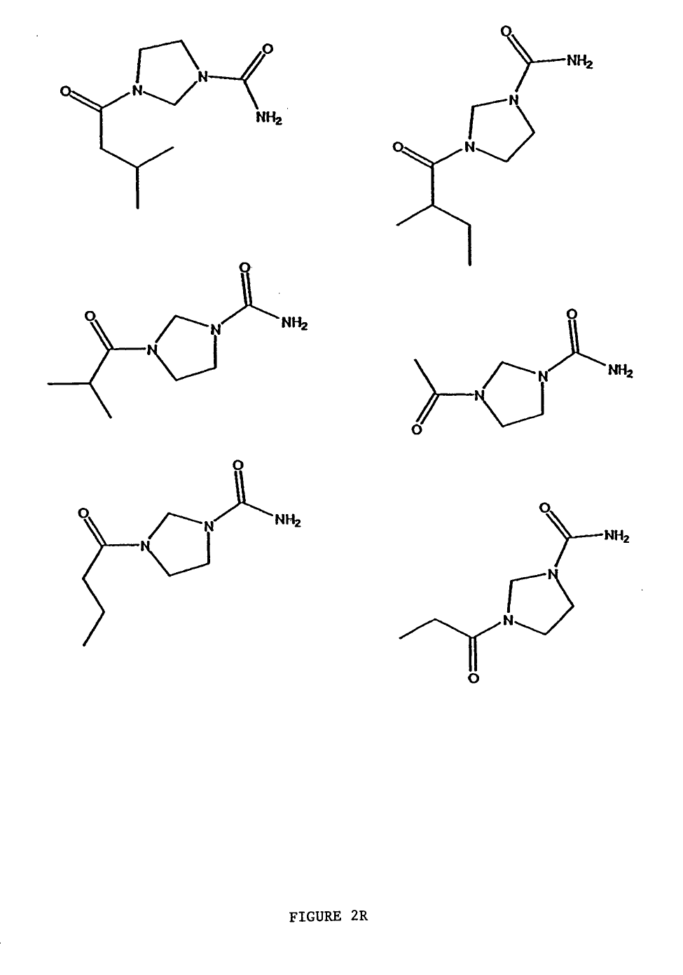

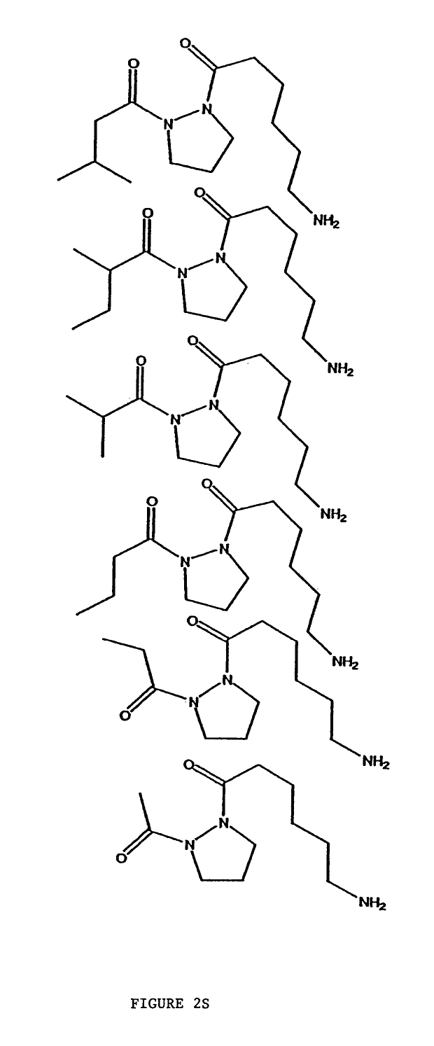

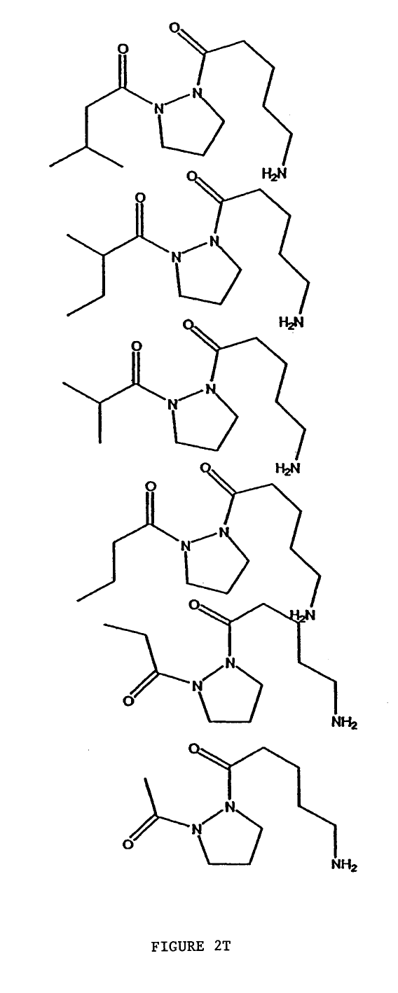

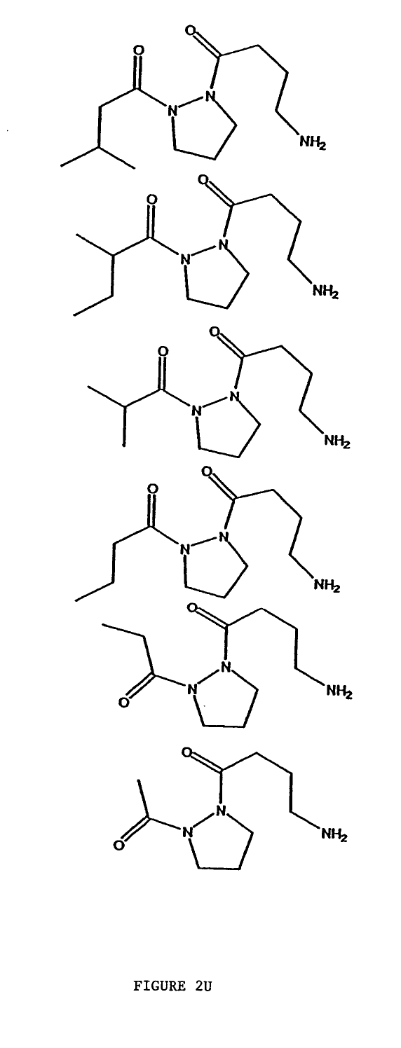

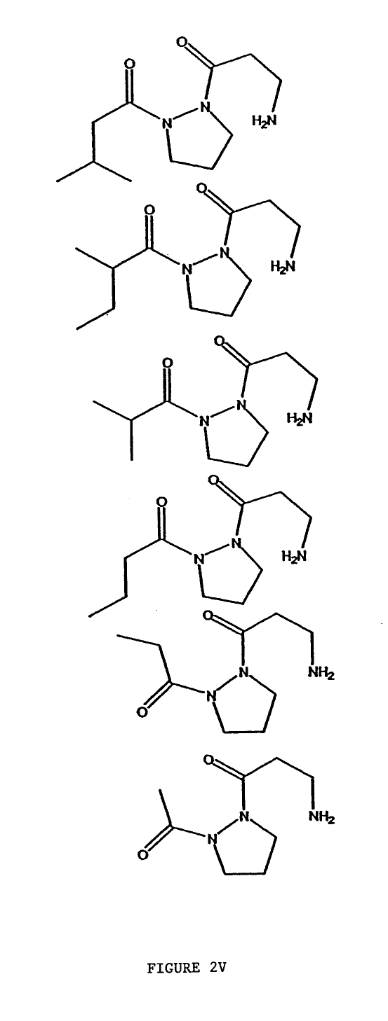

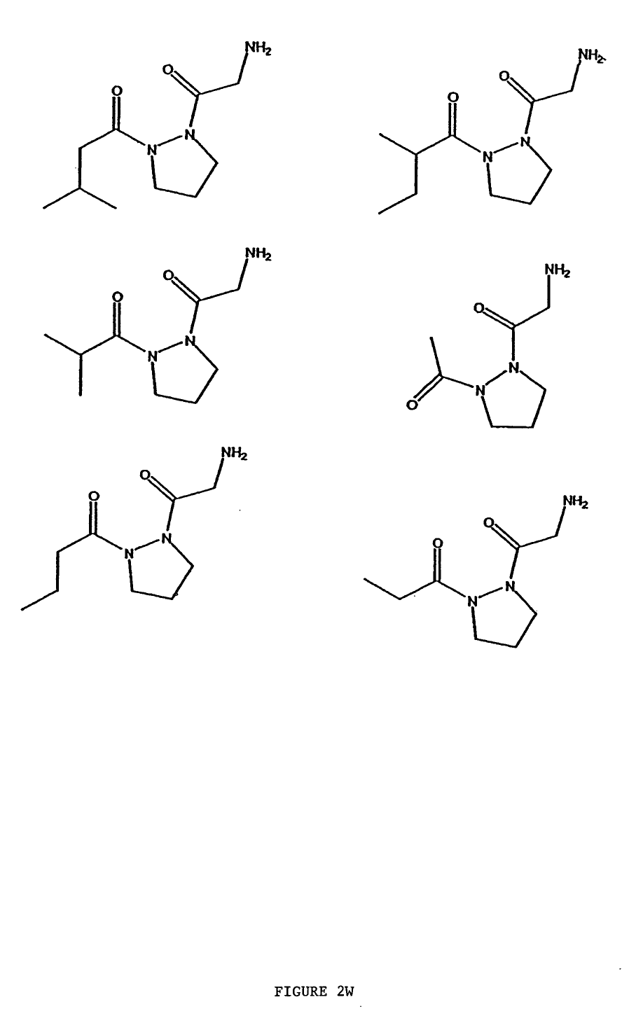

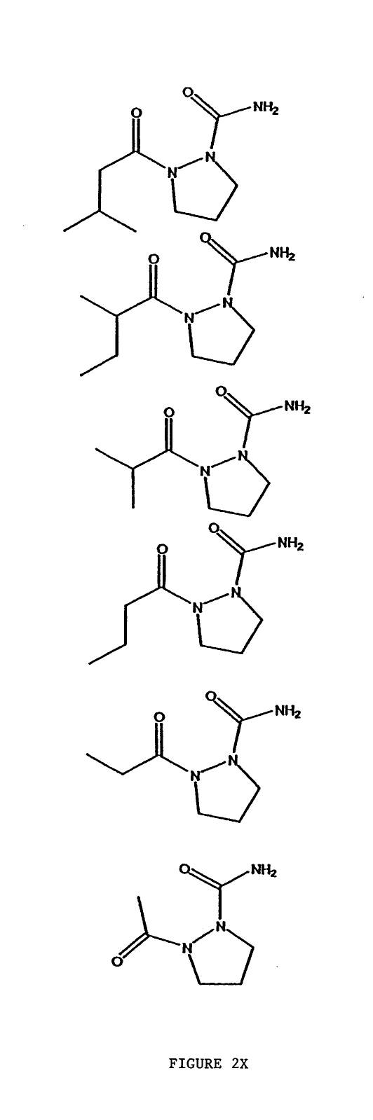

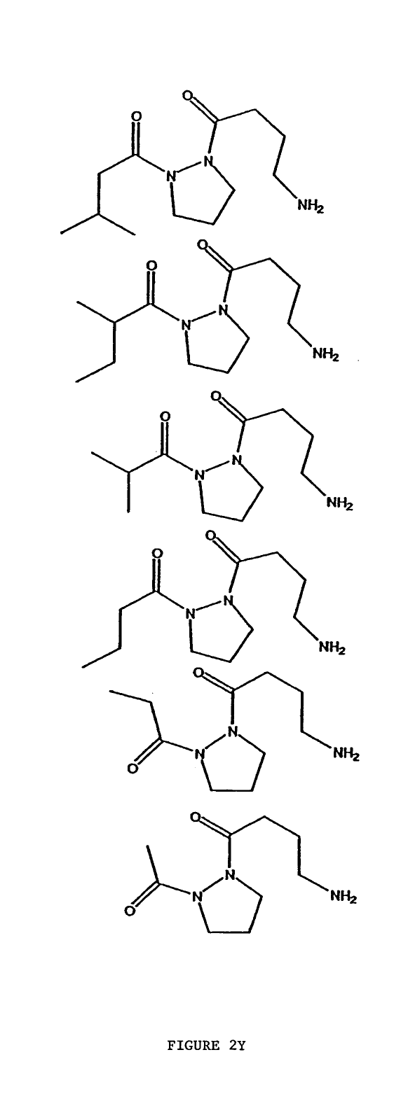

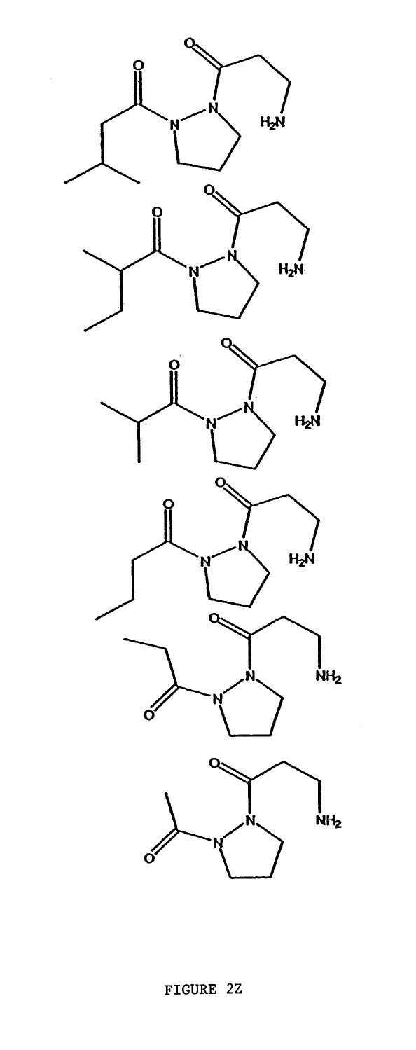

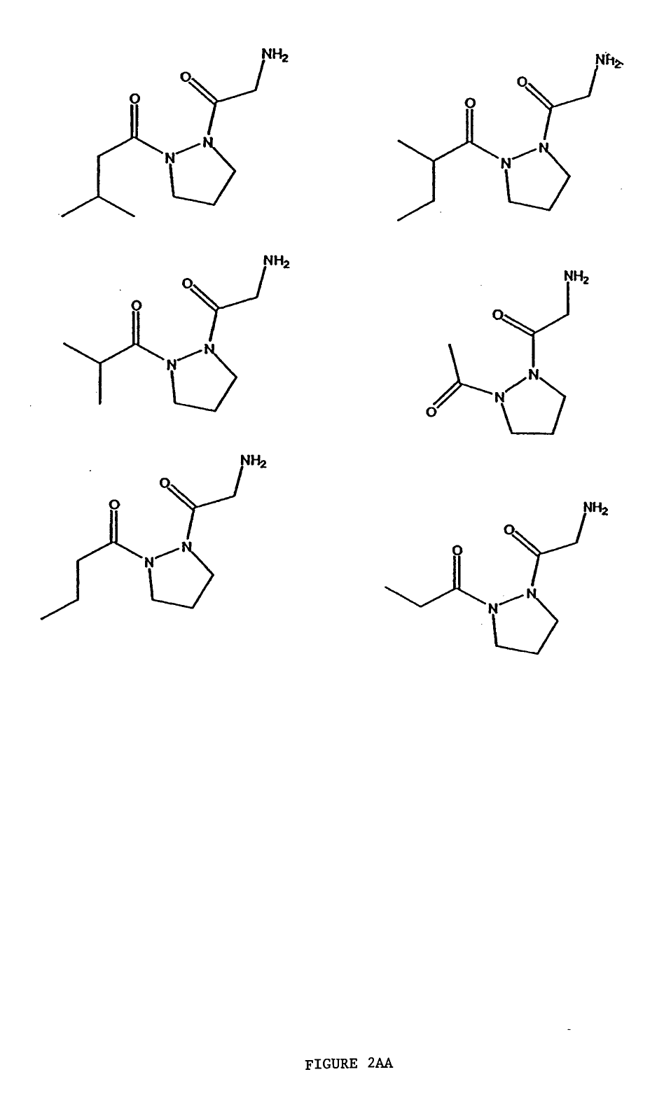

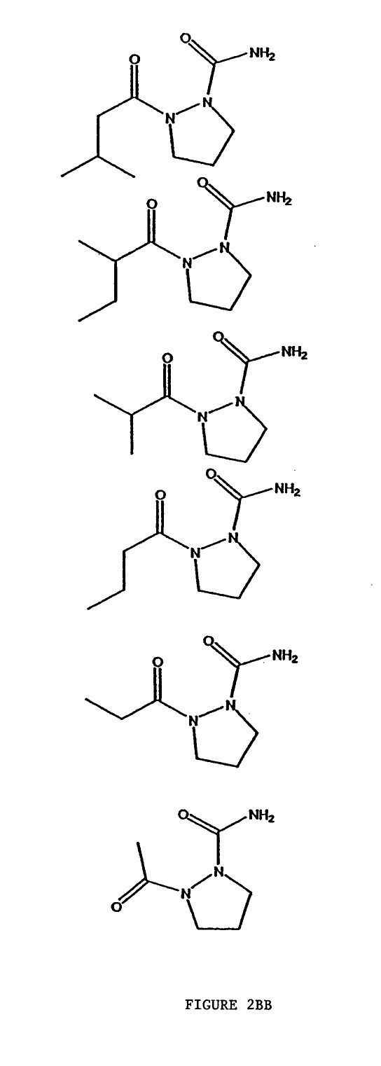

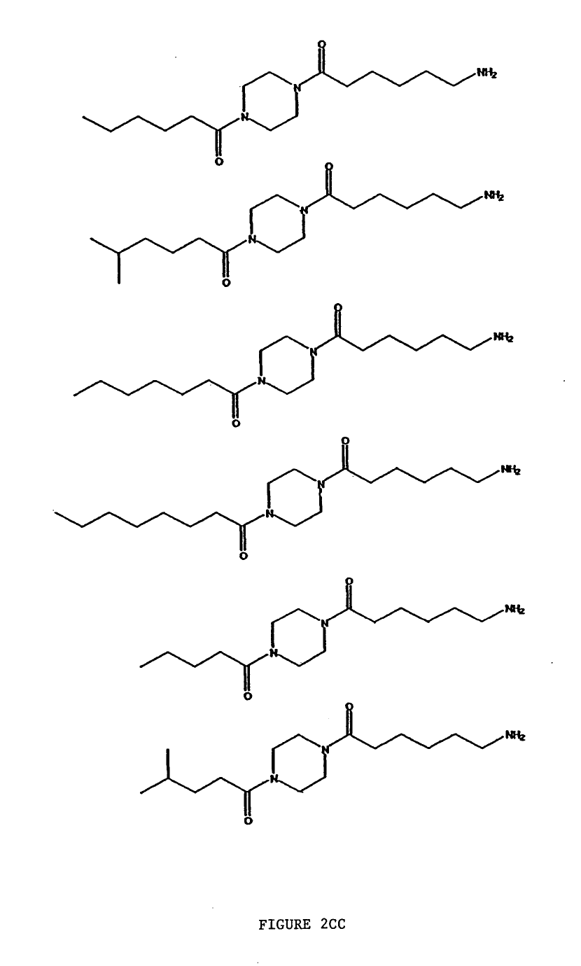

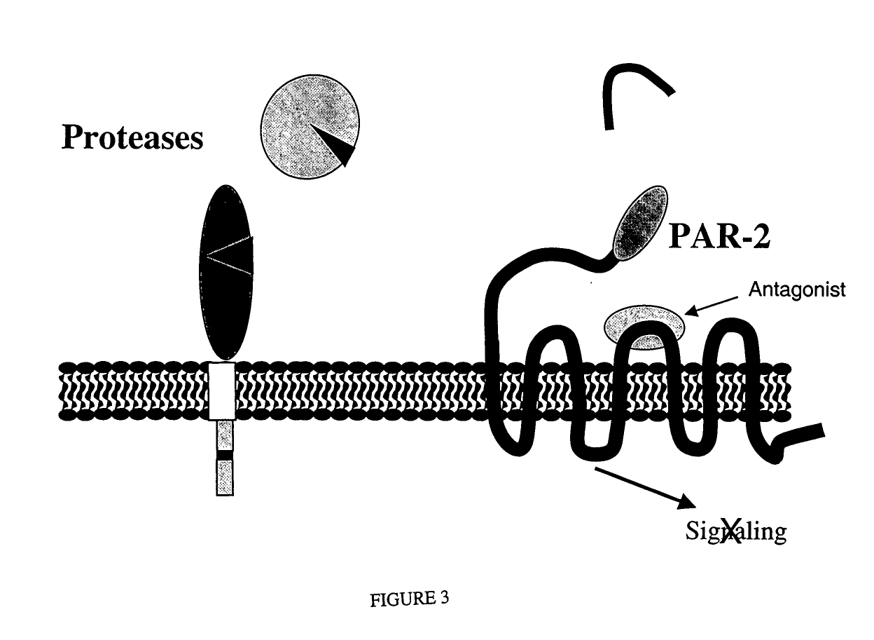

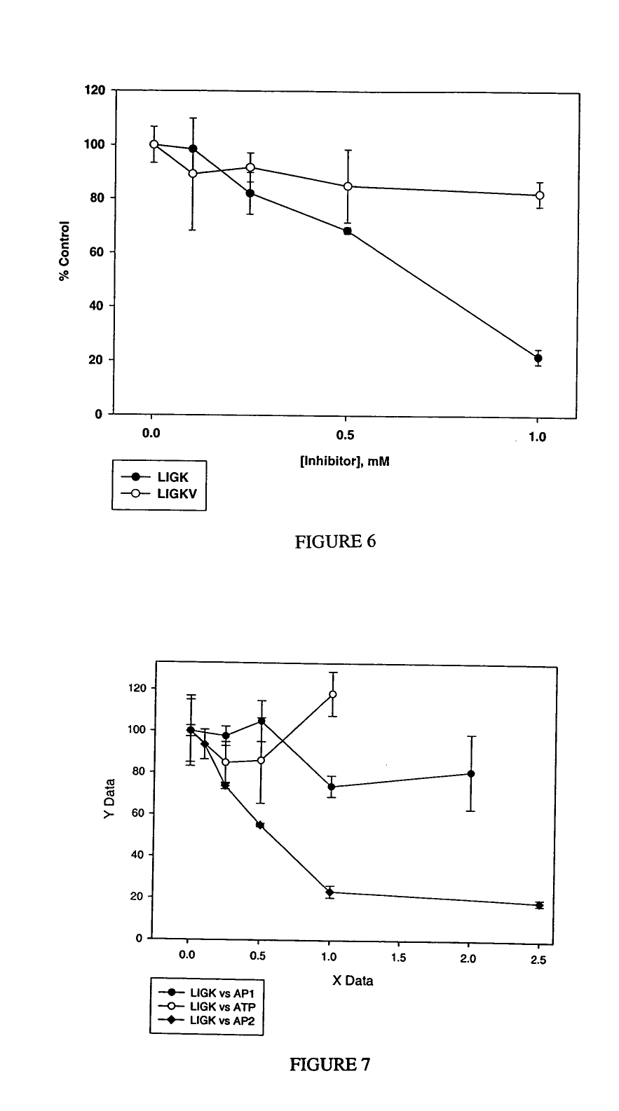

The inventors hypothesized that one of these peptides which lack PAR-2 signaling activity, might function instead as a PAR-2 antagonist, since it would retain the ability to bind to the PAR-2 receptor, while lacking the ability to signal. In this way, such a peptide would function as a competitive inhibitor, since it would block or displace the endogenous agonist peptide from binding and signaling. In order to assess the potential of these peptides to block PAR-2 signaling, cells were pretreated with potential antagonist peptides for a predetermined amount of time and were subsequently treated with P2AP. Two of the SLIGKV (SEQ ID NO:25) derived peptides demonstrated antagonist activity, LIGK (SEQ ID NO:1) and LIGKV (SEQ ID NO:2). FIG. 5 shows a representative dosing study where increasing concentrations of LIGK (SEQ ID NO:1) were used to block P2AP signaling. In this study, a concentration of 1 mM LIGK (SEQ ID NO:1) completely blocked the signaling of 100 uM SLIGKV (SEQ ID NO:25). In similar studies comparing the activity of LIGK (SEQ ID NO:1) with LIGKV (SEQ ID NO:2) it was found that the LIGK (SEQ ID NO:1) peptide is a more potent inhibitor of PAR-2 signaling (IC50<0.5 mM), compared to LIGKV (SEQ ID NO:2) (FIG. 6). Additional peptides include but are not limited to: KGIL (SEQ ID NO:3), KGI (SEQ ID NO:4), AGI (SEQ ID NO:5), IGA (SEQ ID NO:6), KGA (SEQ ID NO:7), KGA (SEQ ID NO:8), KAI (SEQ ID NO:9), IAK (SEQ ID NO:10), RGI (SEQ ID NO:11), IGR (SEQ ID NO:12), Dab-GI (Dab=diamino butanoic acid) (SEQ ID NO:13 ), Dap-GI (Dap=diamino proprionic acid) (SEQ ID NO:14), IG-Dab (SEQ ID NO:15 ), IG-Dap (SEQ ID NO:16), LIG-Dab (SEQ ID NO:17), Dab-GIL (SEQ ID NO:18), LIG-Dap (SEQ ID NO:19), Dap-GIL (SEQ ID NO:20), LIG-Orn (SEQ ID NO:21), Orn-GIL (SEQ ID: 22), Orn-GI (SEQ ID NO:23) and IG-Orn (SEQ ID NO:24), ENMD 545 (FIG. 1), ENMD 547 (FIG. 1), and various peptidomimetic structures provided in FIG. 2.

In order to demonstrate that LIGK (SEQ ID NO:1) is a specific inhibitor of PAR-2 signaling, activation studies were performed with ATP and the PAR-1 activation peptide, SFLLRN (SEQ ID NO:34), on cells that were pretreated with LIGK. Both of these molecules signal through G-protein coupled receptors, and PAR-1 is very highly homologous to PAR-2, to the degree that the PAR-1 agonist peptide can signal through PAR-2 at high concentrations. In both cases, the PAR-2 antagonist LIGK (SEQ ID NO:1) had no inhibitory effect on signaling (FIG. 7).

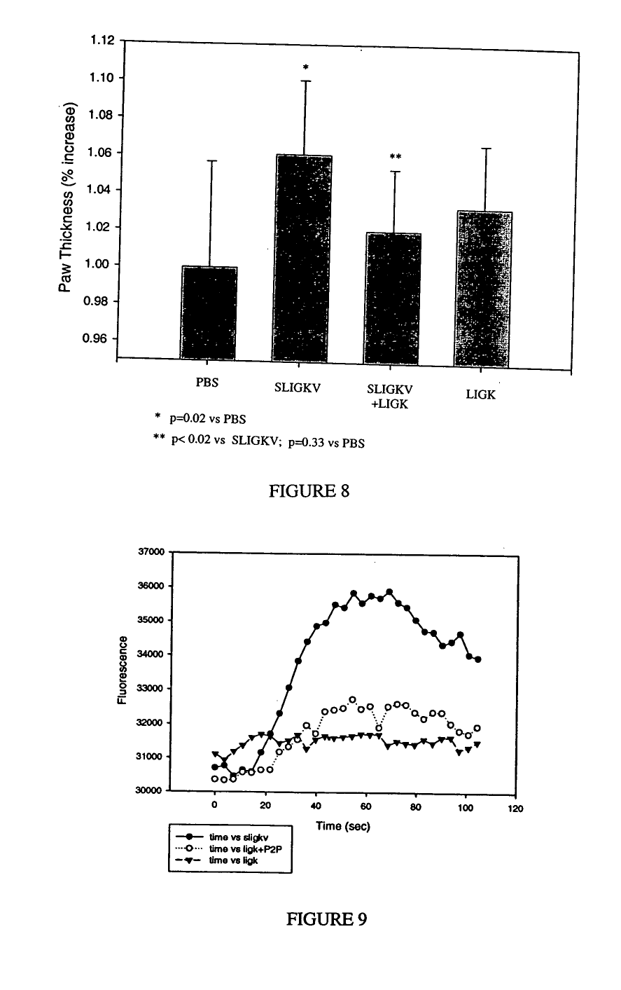

The inventors next assessed whether the LIGK peptide had in vivo PAR-2 antagonistic activity. This was studied using an edema model where vascular permeability was induced by the PAR-2 agonist peptide. In this model, the PAR-2 peptide induces severe edema as expected (FIG. 8). This vascular response was blocked by co-treatment with the PAR-2 antagonist LIGK (FIG. 9). Thus, LIGK functions in vivo to block PAR-2 signaling.

Previous work by the inventors demonstrated that the proteolytic activity of TF/fVIIa promoted angiogenesis and tumor growth through a non-hemostatic mechanism. It was theorized that cleavage of PAR-2 by TF/fVIIa might represent the mechanism whereby TF/fVIIa stimulates these processes. For these reasons, the inventors sought to characterize the ability of LIGK to inhibit tumor growth. PAR-2 activity was first assessed in the Lewis lung carcinoma (LLC) experimental metastatic model.

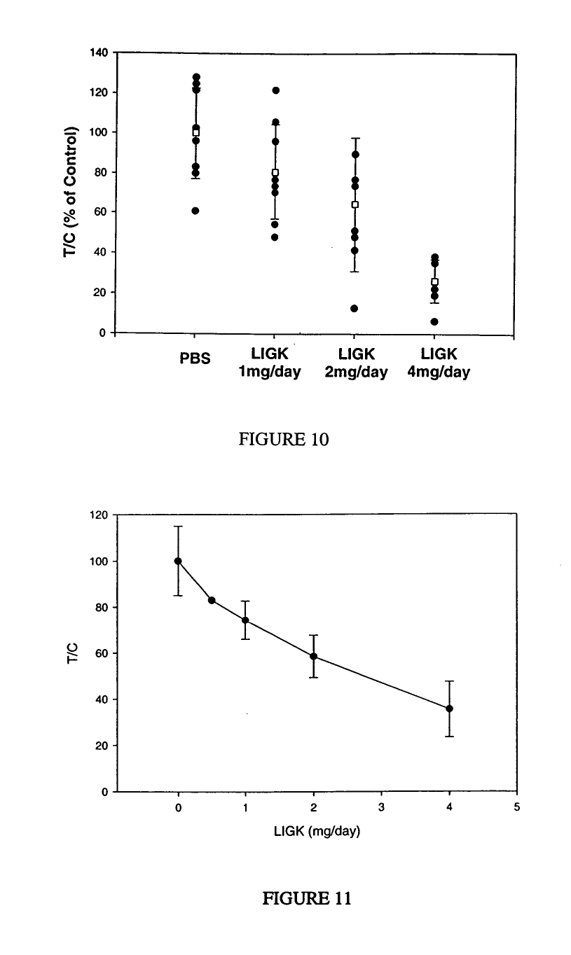

In this tumor growth model, treatments were initiated on day 3 post inoculation, after tumor cells had homed to the lung, and started growing. In this model (FIG. 10), the PAR-2 antagonist LIGK was found to be a very potent inhibitor of metastatic tumor growth. At a dose of 4 mg/day tumor growth was inhibited by 75%. LIGK also demonstrated dose dependency across multiple independent studies, with an approximate IC50 of 2 mg/day (FIG. 11).

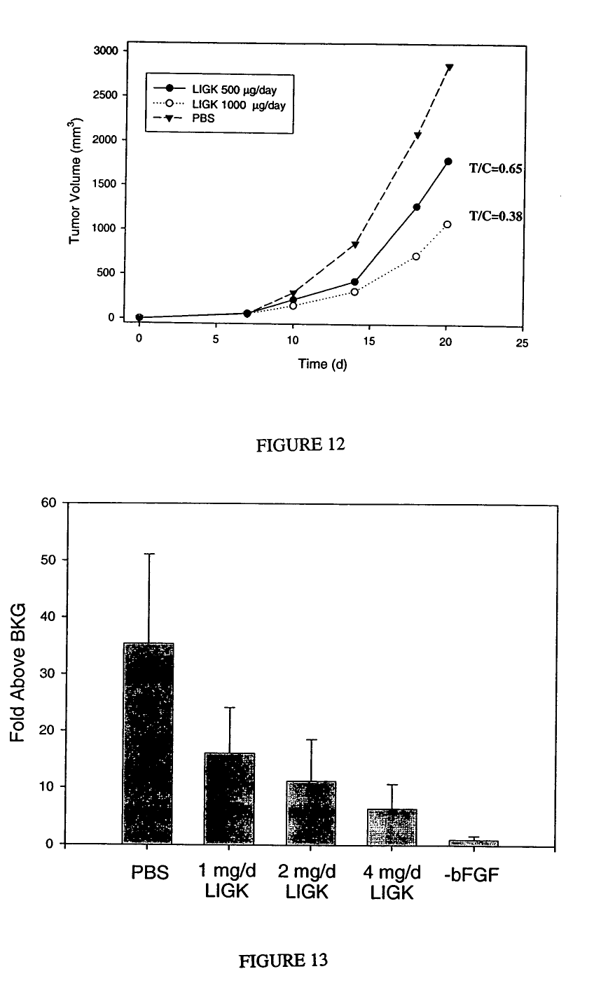

Similar experiments were performed in the LLC primary tumor model. In this model, treatment is initiated when tumor volume approaches 100 mm³. Consistent with the metastasis model, LIGK proved to be a very potent inhibitor of LLC primary tumor (FIG. 12). At 1 mg/day, tumor growth was inhibited by 62%.

Since TF/fVIIa inhibitors are also potent antiangiogenic agents, we tested the antiangiogenic activity of LIGK in the Matrigel angiogenesis model. In this assay Matrigel admixed with bFGF are implanted subcutaneously and treatments are initiated 24 h later. bFGF control plugs are highly vascularized and filled with blood filled vessels. Matrigel plugs from animals treated with LIGK demonstrated a dose dependent inhibition of angiogenesis, based upon hemoglobin content in the plug (FIG. 13). At the highest dose of LIGK, angiogenesis was inhibited by more than 80%. These data demonstrate that LIGK has potent antiangiogenic activity, and further suggest a mechanism by which LIGK could block tumor growth.

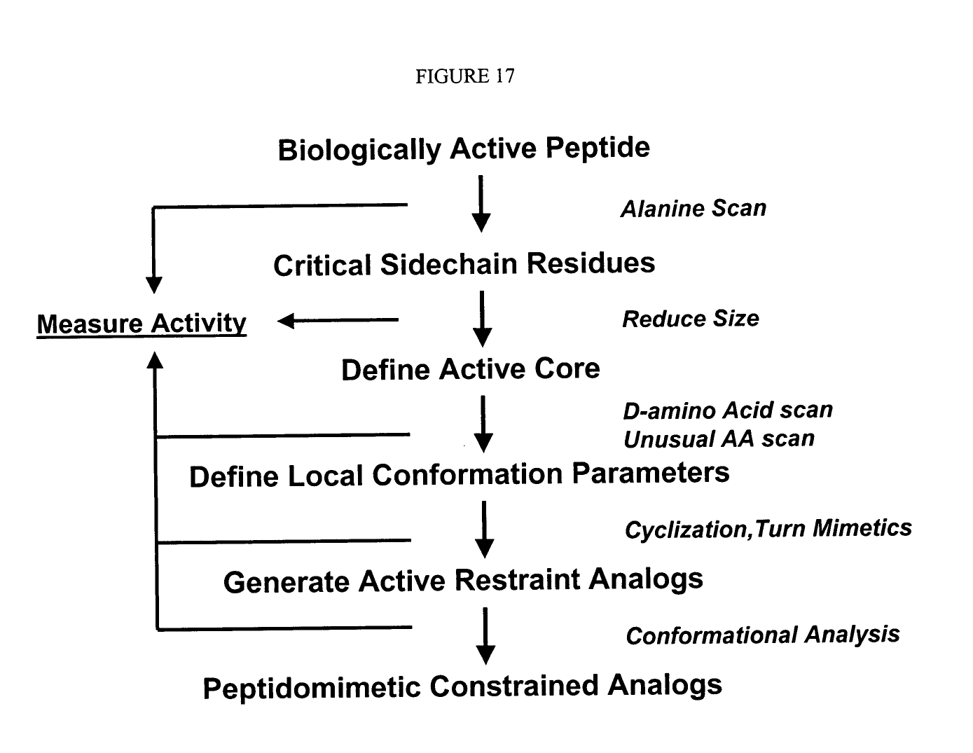

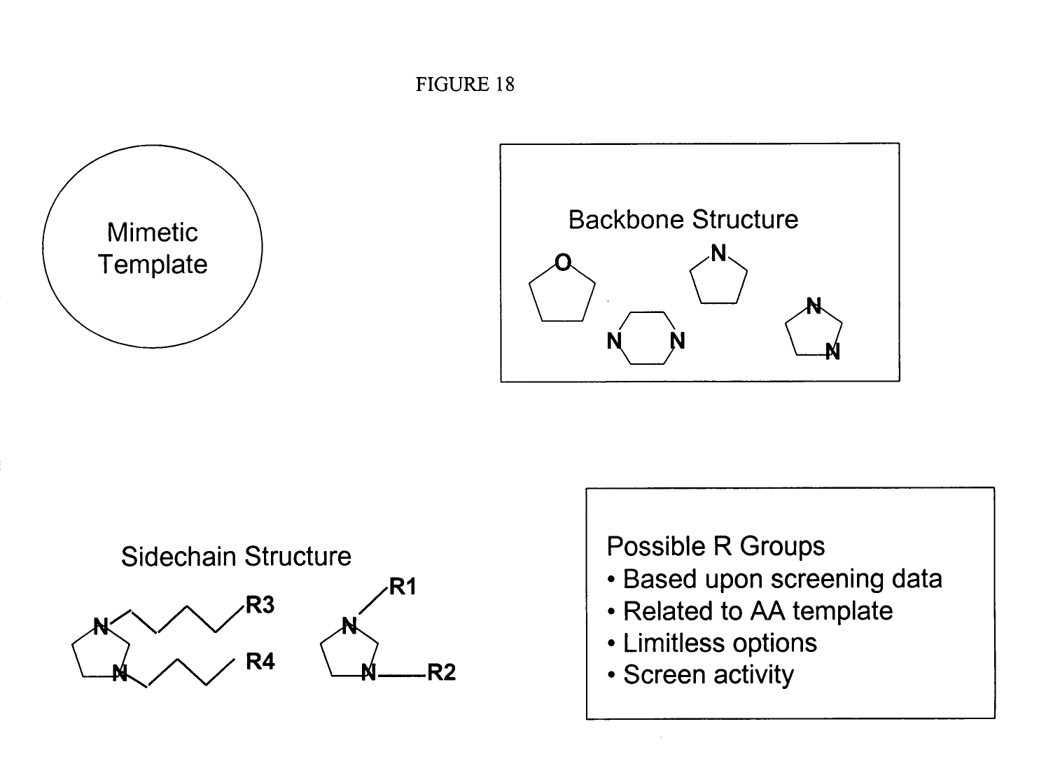

In order to confirm the role PAR-2 in these tumor models, the inventors sought to synthesize novel peptidomimetic antagonists based on the structure of the LIGK antagonist peptide. The structure of these inhibitors was based on the LIGK sequence, generally comprising conformations that have a basic portion one side (for example a lysine) and a linker attaching that side to a hydrophobic portion on the other side. Based on the findings of the present studies, the inventors sought to design non-peptide PAR-2 antagonists that were non-hydrolysable, orally active and simple to synthesize. For certain embodiments, the inventors incorporated molecules mimicking the terminal Leu and Lys from LIGK, and a hydrophobic linker mimicking Ile and Gly in LIGK. A listing of several such structures and biomolecules is provided in FIG. 2. A flow chart showing the peptidomimetic approach taken by the inventors is provided in FIG. 17 and a schematic showing peptidomimetic design is provided in FIG. 18.

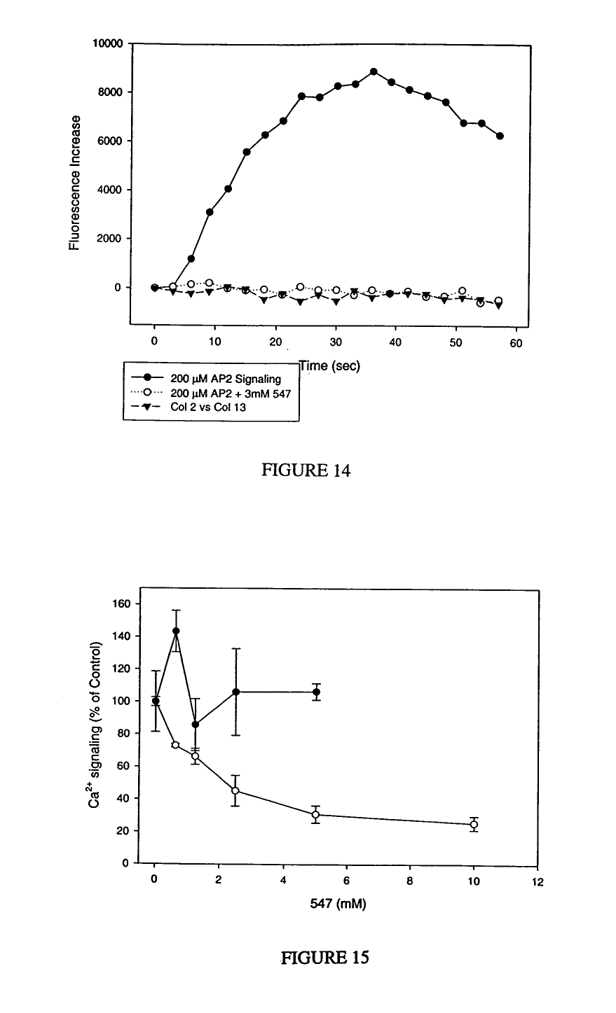

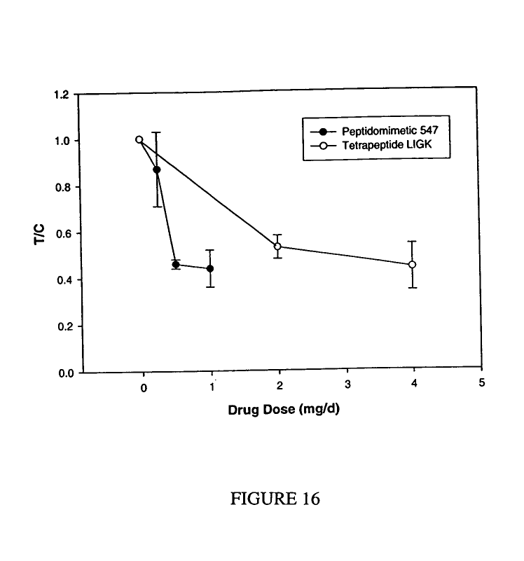

One peptidomimetic antagonist of the LIGK antagonist peptide of particular interest is ENMD-547. The structure of ENMD-547 comprises a piperizine ring to which a 6 amino-hexanoic acid moiety is attached to a nitrogen molecule of the piperizine ring, and a isovaleric acid is attached to the opposite nitrogen (FIG. 1). ENMD-547 was discovered to be an extremely potent inhibitor of PAR-2 signaling in vitro (FIG. 14). Like the LIGK peptide ENMD-547 has no inhibitory effects on signaling by ATP or PAR-1 (not shown). Finally in metastatic tumor growth studies, ENMD-547 has potent antitumor activity, approximately five fold better than the parental LIGK molecule (FIG. 16). Taken together, the identification of a second specific PAR-2 inhibitor with antitumor activity supports the inventors' contention that PAR-2 plays a vital role in the growth and development of tumors in vivo. In addition this molecule, due to its enhanced antitumor activity, may provide insight into the design and synthesis of other PAR-2 antagonist molecules.

These studies, taken together, demonstrate that PAR-2 plays a very important role in the promotion of angiogenesis and tumor growth. Furthermore the inventors demonstrate a very compelling way in which activation of coagulation may promote tumor growth or angiogenesis through a process that is independent of coagulation. Though not wishing to be bound by the following theory, it is thought that the TF/fVIIa complex may be responsible for activating PAR-2 in these angiogenic and tumor models. However, several other proteinases can activate PAR-2, and may promote these novel PAR-2 activities (although LIGK will inhibit activation of PAR-2 independent of the proteinase that activates it). The most relevant enzymes for these processes are mast cell tryptase, trypsin and matriptase. Each of these enzymes undoubtedly plays an important role in PAR-2 physiology, and none can be excluded as from consideration in this specific case. Thus, the TF/fVIIa-PAR-2 pathway is a very strong candidate for the proangiogenic and protumor activities demonstrated here. Specific inhibitors of the TF/fVIIa signaling complex as well as specific inhibitors of the signaling receptor have identical antitumor and antiangiogenic activity. Recent studies on TF demonstrate that this molecule is an immediate early gene that is expressed on angiogenic endothelium. Thus this PAR-2 activator is upregulated and present at the site of angiogenesis. The present studies demonstrating an antiangiogenic activity for LIGK, and the predicted antitumor activity this antiangiogenic activity might have, does not exclude a direct antitumor activity.

It is further possible that there is also a direct antitumor effect of the PAR-2 antagonist molecule on LLC tumor growth. PAR-2 agonists can stimulate tumor cell growth in vitro, and may have similar activity in vivo, though our studies show that LIGK has no antiproliferative effect on LLC in vitro (data not shown). It may be possible to address the question of which compartment the PAR-2 antagonist is acting upon by performing tumor studies on PAR-2 knockout mice, which are challenged with PAR-2 expressing tumors.

The term “active portion” is defined herein as the portion of a ligand or molecule necessary for inhibiting the activity of protein activated receptors. The active portion has the ability to inhibit protein activated receptors expression by in vivo or in vitro assays or other known techniques.

As noted above, the compositions of the present invention may be optionally combined with a pharmaceutical carrier. The term “carrier” as used herein comprises delivery mechanisms known to those skilled in the art including, but not limited to, keyhole limpet hemocyanin (KLH), bovine serum albumin (BSA) and other adjuvants. It is to be understood that the low density lipoprotein receptor ligand compositions of the present invention can further comprise adjuvants, preservatives, diluents, emulsifiers, stabilizers, and other components that are known and used for pharmaceutical compositions of the prior art. Any adjuvant system known in the art can be used for the compositions of the present invention. Such adjuvants include, but are not limited to, Freund's incomplete adjuvant, Freund's complete adjuvant, polydispersed β-(1,4) linked acetylated mannan (“Acemannan”), TITERMAX® (polyoxyethylene-polyoxypropylene copolymer adjuvants from CytRx Corporation (Norcross, Ga.), modified lipid adjuvants from Chiron Corporation (Emeryville, Calif.), saponin derivative adjuvants from Aguila Biopharmaceuticals (Worcester, Mass.), killed Bordetella pertussis, the lipopolysaccharide (LPS) of gram-negative bacteria, large polymeric anions such as dextran sulfate, and inorganic gels such as alum, aluminum hydroxide, or aluminum phosphate, ovalbumin; flagellin; thyroglobulin; serum albumin of any species; gamma globulin of any species; and polymers of D- and/or L-amino acids.

In accordance with the methods of the present invention, the compositions described herein, containing a protein, peptide, or protein fragment including all or an active portion of ligand that binds a blood clotting component, optionally in a pharmaceutically acceptable carrier, is administered to a human or animal exhibiting undesirable cell proliferation in an amount sufficient to inhibit the undesirable cell proliferation, particularly endothelial cell proliferation, angiogenesis or an angiogenesis-related disease, such as cancer.

### DEFINITIONS

The terms “a”, “an” and “the” as used herein are defined to mean one or more and include the plural unless the context is inappropriate.

As used herein, the phrase “protein activated receptor” is defined to encompass all protein activated receptors (PARS), including but not limited to PAR-1, PAR-2, PAR-3 and PAR-4.

The term “antagonist” is used herein to define a protein, peptide or biomolecule that inhibits protein activated receptor activity.

The term “peptides,” are chains of amino acids (typically L-amino acids) whose alpha carbons are linked through peptide bonds formed by a condensation reaction between the carboxyl group of the alpha carbon of one amino acid and the amino group of the alpha carbon of another amino acid. The terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free alpha-amino group on the amino acid at the amino terminal of the peptide, or to the alpha-amino group (imino group when participating in a peptide bond) of an amino acid at any other location within the peptide. Similarly, the term “carboxy terminus” (abbreviated C-terminus) refers to the free carboxyl group on the amino acid at the carboxy terminus of a peptide, or to the carboxyl group of an amino acid at any other location within the peptide.

Typically, the amino acids making up a peptide are numbered in order, starting at the amino terminal and increasing in the direction toward the carboxy terminal of the peptide. Thus, when one amino acid is said to “follow” another, that amino acid is positioned closer to the carboxy terminal of the peptide than the preceding amino acid.

The term “residue” is used herein to refer to an amino acid (D or L) that is incorporated into a peptide by an amide bond. As such, the amino acid may be a naturally occurring amino acid or, unless otherwise limited, may encompass known analogs of natural amino acids that function in a manner similar to the naturally occurring amino acids (i.e., amino acid mimetics). Moreover, an amide bond mimetic includes peptide backbone modifications well known to those skilled in the art.

The phrase “consisting essentially of” is used herein to exclude any elements that would substantially alter the essential properties of the peptides to which the phrase refers. Thus, the description of a peptide “consisting essentially of . . . ” excludes any amino acid substitutions, additions, or deletions that would substantially alter the biological activity of that peptide.

Furthermore, one of skill will recognize that, as mentioned above, individual substitutions, deletions or additions which alter, add or delete a single amino acid or a small percentage of amino acids (typically less than 5%, more typically less than 1%) in an encoded sequence are conservatively modified variations where the alterations result in the substitution of an amino acid with a chemically similar amino acid. Conservative substitution tables providing functionally similar amino acids are well known in the art. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

The phrases “isolated” or “biologically pure” refer to material which is substantially or essentially free from components which normally accompany it as found in its native state. Thus, the peptides described herein do not contain materials normally associated with their in situ environment. Typically, the isolated, antiproliferative peptides described herein are at least about 80% pure, usually at least about 90%, and preferably at least about 95% as measured by band intensity on a silver stained gel.

Protein purity or homogeneity may be indicated by a number of methods well known in the art, such as polyacrylamide gel electrophoresis of a protein sample, followed by visualization upon staining. For certain purposes high resolution will be needed and HPLC or a similar means for purification utilized.

When the inhibitory peptides are relatively short in length (i.e., less than about 50 amino acids), they are often synthesized using standard chemical peptide synthesis techniques.

Solid phase synthesis in which the C-terminal amino acid of the sequence is attached to an insoluble support followed by sequential addition of the remaining amino acids in the sequence is a preferred method for the chemical synthesis of the antiproliferative peptides described herein. Techniques for solid phase synthesis are known to those skilled in the art.

Alternatively, the inhibitory peptides described herein are synthesized using recombinant nucleic acid methodology. Generally, this involves creating a nucleic acid sequence that encodes the peptide, placing the nucleic acid in an expression cassette under the control of a particular promoter, expressing the peptide in a host, isolating the expressed peptide or polypeptide and, if required, renaturing the peptide. Techniques sufficient to guide one of skill through such procedures are found in the literature.

Once expressed, recombinant peptides can be purified according to standard procedures, including ammonium sulfate precipitation, affinity columns, column chromatography, gel electrophoresis and the like. Substantially pure compositions of about 50 to 95% homogeneity are preferred, and 80 to 95% or greater homogeneity are most preferred for use as therapeutic agents.

One of skill in the art will recognize that after chemical synthesis, biological expression or purification, the antiproliferative peptides may possess a conformation substantially different than the native conformations of the constituent peptides. In this case, it is often necessary to denature and reduce the antiproliferative peptide and then to cause the peptide to re-fold into the preferred conformation. Methods of reducing and denaturing proteins and inducing re-folding are well known to those of skill in the art.

As employed herein, the phrase “biological activity” refers to the functionality, reactivity, and specificity of compounds that are derived from biological systems or those compounds that are reactive to them, or other compounds that mimic the functionality, reactivity, and specificity of these compounds. Examples of suitable biologically active compounds include enzymes, antibodies, antigens and proteins.

The term “bodily fluid,” as used herein, includes, but is not limited to, saliva, gingival secretions, cerebrospinal fluid, gastrointestinal fluid, mucous, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, and vitreal fluid, and nasal secretions.

The inhibitory proteins and peptides of protein activated receptors of the present invention may be isolated from body fluids including, but not limited to, serum, urine, and ascites, or may be synthesized by chemical or biological methods, such as cell culture, recombinant gene expression, and peptide synthesis. Recombinant techniques include gene amplification from DNA sources using the polymerase chain reaction (PCR), and gene amplification from RNA sources using reverse transcriptase/PCR. Ligands of interest are extracted from body fluids by known protein extraction methods, particularly the method described by Novotny, W. F., et al., J. Biol. Chem. 264:18832-18837 (1989).

Peptides or Protein Fragments

Peptides or protein fragments comprising PAR antagonists can be produced as described above and tested for inhibitory activity using techniques and methods known to those skilled in the art. Full length proteins can be cleaved into individual domains or digested using various methods such as, for example, the method described by Enjyoji et al. (Biochemistry 34:5725-5735 (1995)).

Alternatively, fragments are prepared by digesting the entire protein, or large fragments thereof exhibiting anti-proliferative activity, to remove one amino acid at a time. Each progressively shorter fragment is then tested for anti-proliferative activity. Similarly, fragments of various lengths may be synthesized and tested for inhibitory activity. By increasing or decreasing the length of a fragment, one skilled in the art may determine the exact number, identity, and sequence of amino acids within the protein that are required for inhibitory activity using routine digestion, synthesis, and screening procedures known to those skilled in the art.

Inhibitory activity is evaluated in situ by testing the ability of the proteins and peptides to inhibit the activation of PAR. Suitable assays are well known to skilled in the art and several examples of such are provided below in the Examples. Antiangiogenic activity may be assessed using the chick embryo chorioallantoic membrane (CAM) assay described by Crum et al., Science 230:1375 (1985) and described in U.S. Pat. No. 5,001,116, which is incorporated by reference herein. The CAM assay is briefly described as follows. Fertilized chick embryos are removed from their shell on day 3 or 4, and a methylcellulose disc containing the fragment of interest is implanted on the chorioallantoic membrane. The embryos are examined 48 hours later and, if a clear avascular zone appears around the methylcellulose disc, the diameter of that zone is measured. The larger the diameter of the zone, the greater the anti-angiogenic activity. Another suitable assay is the HUVEC assay.

As discussed above, one of skill in the art will recognize that, individual substitutions, deletions or additions which alter, add or delete a single amino acid or a small percentage of amino acids (typically less than 5%, more typically less than 1%) in an encoded sequence are conservatively modified variations where the alterations result in the substitution of an amino acid with a chemically similar amino acid. Conservative substitution tables providing functionally similar amino acids are well known in the art. Accordingly, also included in the present invention are peptides having conservatively modified variations in comparison to the claimed peptides, wherein the chemical reactivity of the peptide is not significantly different from that of the claimed peptide.

Formulations

The naturally occurring or synthetic protein, peptide, or protein fragment, containing all or an active portion of a protein, peptide or biomolecule that may bind to a protein activated receptor can be prepared in a physiologically acceptable formulation, such as in a pharmaceutically acceptable carrier, using known techniques. For example, the protein, peptide, protein fragment or biomolecule is combined with a pharmaceutically acceptable excipient to form a therapeutic composition.

Alternatively, the gene for the protein, peptide, or protein fragment, containing all or an active portion of a desired ligand, may be delivered in a vector for continuous administration using gene therapy techniques. The vector may be administered in a vehicle having specificity for a target site, such as a tumor.

The composition may be in the form of a solid, liquid or aerosol. Examples of solid compositions include pills, creams, and implantable dosage units. Pills may be administered orally. Therapeutic creams may be administered topically. Implantable dosage units may be administered locally, for example, at a tumor site, or may be implanted for systematic release of the therapeutic composition, for example, subcutaneously. Examples of liquid compositions include formulations adapted for injection subcutaneously, intravenously, intra-arterially, and formulations for topical and intraocular administration. Examples of aerosol formulations include inhaler formulations for administration to the lungs.

The composition may be administered by standard routes of administration. In general, the composition may be administered by topical, oral, rectal, nasal or parenteral (for example, intravenous, subcutaneous, or intermuscular) routes. In addition, the composition may be incorporated into sustained release matrices such as biodegradable polymers, the polymers being implanted in the vicinity of where delivery is desired, for example, at the site of a tumor. The method includes administration of a single dose, administration of repeated doses at predetermined time intervals, and sustained administration for a predetermined period of time.

A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The dosage of the composition will depend on the condition being treated, the particular composition used, and other clinical factors such as weight and condition of the patient, and the route of administration.

Further, the term “effective amount” refers to the amount of the composition which, when administered to a human or animal, inhibits protein activated receptor activity, particularly undesirable cell proliferation, causing a reduction in cancer or inhibition in the spread and proliferation of cancer. The effective amount is readily determined by one of skill in the art following routine procedures.

For example, inhibitory compositions of the present invention may be administered parenterally or orally in a range of approximately 1.0 μg to 1.0 mg per patient, though this range is not intended to be limiting. The actual amount of inhibitory composition required to elicit an appropriate response will vary for each individual patient depending on the potency of the composition administered and on the response of the individual. Consequently, the specific amount administered to an individual will be determined by routine experimentation and based upon the training and experience of one skilled in the art.

The composition may be administered in combination with other compositions and procedures for the treatment of diseases. For example, unwanted cell proliferation may be treated conventionally with surgery, radiation or chemotherapy in combination with the administration of the composition, and additional doses of the composition may be subsequently administered to the patient to stabilize and inhibit the growth of any residual unwanted cell proliferation.

Antibodies of Protein Activated Receptor Antagonists

The present invention further comprises antibodies of PAR antagonists that may be used for diagnostic as well as therapeutic purposes. The antibodies provided herein are monoclonal or polyclonal antibodies having binding specificity for desired ligands. The preferred antibodies are monoclonal antibodies, due to their higher specificity for the ligands. The antibodies exhibit minimal or no crossreactivity with other proteins or peptides. Preferably, the antibodies are specific for peptides comprising LIGK (SEQ ID NO:1), LIGKV (SEQ ID NO:2), ENMD 545, and ENMD 547. Also included are antibodies generated against protein activated receptor ligands such as AP2.

Monoclonal antibodies are prepared by immunizing an animal, such as a mouse or rabbit, with a whole or immunogenic portion of a desired peptide, such as LIGK (SEQ ID NO:1). Spleen cells are harvested from the immunized animals and hybridomas generated by fusing sensitized spleen cells with a myeloma cell line, such as murine SP2/O myeloma cells (ATCC, Manassas, Va.). The cells are induced to fuse by the addition of polyethylene glycol. Hybridomas are chemically selected by plating the cells in a selection medium containing hypoxanthine, aminopterin and thymidine (HAT).

Hybridomas are subsequently screened for the ability to produce monoclonal antibodies against ligands. Hybridomas producing antibodies that bind to the ligands are cloned, expanded and stored frozen for future production. The preferred hybridoma produces a monoclonal antibody having the IgG isotype, more preferably the IgG1 isotype.

The polyclonal antibodies are prepared by immunizing animals, such as mice or rabbits with a ligand such as antithrombin as described above. Blood sera is subsequently collected from the animals, and antibodies in the sera screened for binding reactivity against the ligand, preferably the antigens that are reactive with the monoclonal antibody described above.

Either the monoclonal antibodies or the polyclonal antibodies, or both may be labeled directly with a detectable label for identification and quantitation of ligands in a biological as described below. Labels for use in immunoassays are generally known to those skilled in the art and include enzymes, radioisotopes, and fluorescent, luminescent and chromogenic substances including colored particles, such as colloidal gold and latex beads. The antibodies may also be bound to a solid phase to facilitate separation of antibody-antigen complexes from non-reacted components in an immunoassay. Exemplary solid phase substances include, but are not limited to, microtiter plates, test tubes, magnetic, plastic or glass beads and slides. Methods for coupling antibodies to solid phases are well known to those skilled in the art.

Alternatively, the antibodies may be labeled indirectly by reaction with labeled substances that have an affinity for immunoglobulin, such as protein A or G or second antibodies. The antibodies may be conjugated with a second substance and detected with a labeled third substance having an affinity for the second substance conjugated to the antibody. For example, the antibodies may be conjugated to biotin and the antibody-biotin conjugate detected using labeled avidin or streptavidin. Similarly, the antibodies may be conjugated to a hapten and the antibody-hapten conjugate detected using labeled anti-hapten antibody. These and other methods of labeling antibodies and assay conjugates are well known to those skilled in the art.

Sensitive immunoassays employing one or more of the antibodies described above are provided by the present invention. The immunoassays are useful for detecting the presence or amount of ligands in a variety of samples, particularly biological samples, such as human or animal biological fluids. The samples may be obtained from any source in which the ligands may exist. For example, the sample may include, but is not limited to, blood, saliva, semen, tears, and urine.

The antibody-antigen complexes formed in the immunoassays of the present invention are detected using immunoassay methods known to those skilled in the art, including sandwich immunoassays and competitive immunoassays. The antibody-antigen complexes are exposed to antibodies similar to those used to capture the antigen, but which have been labeled with a detectable label. Suitable labels include: chemiluminescent labels, such as horseradish peroxidase; electrochemiluminescent labels, such as ruthenium and aequorin; bioluminescent labels, such as luciferase; fluorescent labels such as FITC; and enzymatic labels such as alkaline phosphatase, β-galactosidase, and horseradish peroxidase.

The labeled complex is then detected using a detection technique or instrument specific for detection of the label employed. Soluble antigen or antigens may also be incubated with magnetic beads coated with non-specific antibodies in an identical assay format to determine the background values of samples analyzed in the assay.

Diseases and Conditions to be Treated

The methods and compositions described herein are useful for treating human and animal diseases and processes mediated by abnormal or undesirable cellular proliferation, particularly abnormal or undesirable endothelial cell proliferation, including, but not limited to, hemangioma, solid tumors, leukemia, metastasis, telangiectasia psoriasis scleroderma, pyogenic granuloma, myocardial angiogenesis, plaque neovascularization, coronary collaterals, ischemic limb angiogenesis, corneal diseases, rubeosis, neovascular glaucoma, diabetic retinopathy, retrolental fibroplasia, arthritis, diabetic neovascularization, macular degeneration, wound healing, peptic ulcer, fractures, keloids, vasculogenesis, hematopoiesis, ovulation, menstruation, and placentation. The method and composition are particularly useful for treating angiogenesis-related disorders and diseases by inhibiting angiogenesis.

The methods and compositions described herein are particularly useful for treating cancer, arthritis, macular degeneration, and diabetic retinopathy. Administration of the compositions to a human or animal having prevascularized metastasized tumors is useful for preventing the growth or expansion of such tumors.

The methods and compositions of this invention include the following diseases: abnormal growth by endothelial cells, acne rosacea, acoustic neuroma, adhesions, angiofibroma, arteriovenous malformations, artery occlusion, arthritis, asthma, atherosclerosis, capillary proliferation within plaques, atherosclerotic plaques, atopic keratitis, bacterial ulcers, bartonelosis, Bechet's disease, benign tumors (for example: hemangiomas, acoustic neuromas, neurofibromas, trachomas, pyogenic granulomas), see also neurofibromas and hemangiomas, benign, premalignant and malignant vulvar lesions, best's disease, bladder cancers, block implantation of a blastula, block menstruation (induce amenorrhea), block ovulation, blood-borne tumors, such as leukemias, and neoplastic diseases of the bone marrow; bone marrow, any of various acute or chronic neoplastic diseases of the bone marrow, in which unrestrained proliferation of white blood cells occurs; (also multiple myeloma), bone growth and repair, breast cancer, burns, hypertrophy following, cancer including: solid tumors: rhabdomyosarcomas, retinoblastoma, Ewing's sarcoma, neuroblastoma, osteosarcoma, blood-borne tumors: leukemias, neoplastic diseases of the bone marrow; multiple myeloma diseases, hemangiomas, carotid artery obstruction (carotid obstructive disease) (general, see separate references relating to ocular obstruction), carotid artery obstruction (carotid obstructive disease) (ocular, see separate references relating to general obstruction), carotid obstructive disease, see carotid artery obstruction, central nervous system malignancy, certain immune reactions, see immune disorders/reactions, cervical cancers, chemical burns, cholesteatoma, especially of the middle ear, choroidal neovascularization. choroiditis, chronic or acute inflammation, chronically exercised muscle, cirrhotic liver, contact lens overwear, corneal diseases, corneal graft neovasularization, corneal graft rejection, corneal neovascularization diseases (including, but not limited to: epidemic keratoconjunctivitis, Vitamin A deficiency, contact lens overwear, atopic keratitis, superior limbic keratitis, and pterygium keratitis sicca), corpus luteum formation, Crohn's disease, delayed wound healing, see wound healing, diabetes, diabetic (proliferative) retinopathy, diseases caused by the abnormal proliferation of fibrovascular or fibrous tissue, including all forms of prolific vitreoretinopathy, Eales disease, embryo development, empyema of the thorax, endometriosis, endometrium, epidemic keratoconjuctivitis, Ewing's sarcoma, excessive or abnormal stimulation of endothelial cells, such as: atherosclerosis, eye-related diseases (including: rubeosis (neovascularization of the angle), abnormal proliferation of fibrovascular or fibrous tissue, including all forms of prolific vitreoretinopathy.), female reproductive system: neovascularization of ovarian follicles, corpus luteum, and maternal decisua; female reproductive system: neovascularization of ovarian follicles, corpus luteum, repair of endometrial vessels, angiogenesis in embryonic implantation sites (ovarian hyperstimulation syndromes); female reproductive system, normal angiogenesis: embryonic development, folliculogenesis, luteogenesis, normal menstruating endometrium, fibrinolysis, fibroplasias (see also retrolental and excessive repair in would healing), fibrosing alveolitis, fungal ulcers, gastrointestinal infections, peptic ulcer, ulcerative colitis, Crohn's disease, inflammed polyps, intestinal graft-vs-host reaction, neoplastic tumors, mastocytosis, intestinal ischemia, glaucoma, neovascular, gout or gouty arthritis, graft versus host rejection (see also chronic and acute rejection), granulation tissue of healing wounds, granulations-burns, haemangiomatoses (systemic forms of hemangiomas), hand foot and mouth disease, hair growth, hemangioma, hemophiliac joints, hereditary diseases (such as: Osler-Weber-Rendu disease, hereditary hemorrhagic telangiectasia), Herpes simplex, Herpes zoster, HHT (hereditary hemorrhagic telangiectasia), Osler-Weber-Rendu disease, hypertrophic scars, hypertrophy following surgery, burns and injury, hyperviscosity syndromes, immune disorders, immune reactions, implantation of embryo (2-8 weeks, must mean blastula), infections causing retinitis, see retinitis, infectious diseases caused by microorganisms, inflammation see “chronic inflammation”, inflammatory disorders immune and non-immune, inflammatory reactions, inflammed jonts, Kaposi's sarcoma, leprosy, leukemias, Lewis lung, lipid degeneration (lipid keratopathy), lipoma, lung cancer, lupus (lupus erythematosis, systemic lupus erythematosis), lyme disease, macular degeneration, age-related (subretinal neovascularization), marginal keratolysis, melanoma; B-12 melanoma, meningiomas, mesothelioma, metastasis, tumor, Mooren's ulcer, mycobacteria diseases, myeloma, multiple myeloma diseases, myopia, neoplasias, neoplastic diseases of the bone marrow (any of various acute or chronic) in which unrestrained proliferation of white blood cells occurs, which are blood-borne tumors, including: leukemias, neovascular glaucoma→see glaucoma, neovascular, neovascularization of the angle, neuroblastoma, neurofibroma, neurofibromatosis, neurofibrosarcoma, non-union fractures, ocular angiogenic diseases (such as: diabetic retinopathy, retinopathy of prematurity (retrolental fibroplasic), macular degeneration, corneal graft rejection, neovascular glaucoma, Osler Weber syndrome (Osler-Weber-Rendu disease)), ocular histoplasmosis, presumed, ocular neovascular disease (is involved in approximately twenty eye diseases), ocular tumors, optic pits, oral cancers, Osler-Weber syndrome (Osler-Weber-Rendu disease or HHT (hereditary hemorrhagic telangiectasia)), osteoarthritis, osteomyelitis, osteosarcoma, Paget's disease (osteitis deformans), parasitic diseases, pars planitis, pemphigold, phlyctenulosis, polyarteritis, post-laser complications, proliferation of white blood cells, any of various acute or chronic neoplastic diseases of the bone marrow, in which unrestrained proliferation of white blood cells occurs, see blood-borne tumors, prolific vitreoretinopathy (PVR), prostate cancer, protozoan infections, pseudoxanthoma elasticum, psoriasis, pterygium (keratitis sicca), pulmonary fibrosis, pyogenic granuloma, radial keratotomy, rejection, chronic and acute (see also graft vs. host rejection), retinal detachment (chronic), retinitis, infections causing, retinoblastoma, retinopathy of prematurity, retrolental fibroplasias, rhabdomyosarcomas, rheumatoid arthritis, rheumatoid synovial hypertrophy (arthritis), rosacea (acne rosacea), rubeosis [iris], sarcoidosis, scleritis, scleroderma, sicca, see pterygium (keratitis sicca) and Sjogren's (sicca) syndrome, sickle cell anemia, Sjogren's (sicca) syndrome, skin disease: see also melanoma, pyogenic granulomas, psoriasis and hemangioma, skin warts and HPV type 2 (human papillomavirus), solid tumors (application includes list: rhabdomyosarcomas, retinoblastoma, Ewing's sarcoma, neuroblastoma, osteosarcoma), stargard's disease, Stevens-Johnson's disease, superior limbic keratitis (superior limbic keratoconjuctivitis, SLK), surgery: hypertrophic scars, wound granulation and vascular adhesions, syphilis, systemic lupus, systemic lupus erythematosis, Terrien's marginal degeration, toxoplasmosis, trachoma, trauma, tuberculosis, tumors, tumor associated angiogenesis, tumor growth, ulcerative colitis, ulcers (such as, fungal, Mooren's, peptic and bacterial), undesired angiogenesis in normal processes, such as wound healing, female reproductive functions, bone repair, hair growth, uveitis, chronic, vascular malfunction, vascular tumors, vein occlusion, vitamin A deficiency, vitritis, chronic, Wegener's sarcoidosis, white blood cells, any of various acute or chronic neoplastic diseases of the bone marrow, in which unrestrained proliferation of white blood cells occurs, see blood-borne tumors, wound healing and inappropriate wound healing, delayed wound healing, angiofibroma, arteriovenous malformations, arthritis, atherosclerotic plaques, corneal graft neovascularization, delayed wound healing, diabetic retinopathy, granulations-burns, hemangioma, hemophilic joints, hypertrophic scars, neovascular glaucoma, non-union fractures, Osler-Weber syndrome, psoriasis, pyogenic granuloma, retrolental fibroplasias, scleroderma, solid tumors, trachoma, corpus luteum formations, wound healing, chronically exercised muscle, psoriasis, diabetic retinopathy, tumor vascularization, rheumatoid arthritis, psoriasis, solid tumors, chronic inflammatory diseases, inflamed joints, rheumatoid synovial hypertrophy (arthritis), atherosclerosis, proliferative (diabetic) retinopathy, solid tumors (chronic inflammatory diseases), tumor growth, metastasis, oral cancers, cervical cancers, bladder and breast cancers, melanomas, pyogenic granulomas, tumors, diabetic retinopathy, psoriasis, rheumatoid arthritis, Lewis Lung, B-12 melanoma and haemangiomatoses; follicles mature to corpus luteum, endometrium; Kaposi's sarcoma, wound healing, adhesion, tumor growth, acute and/or chronic inflammation and inflammatory reactions, chronic and acute rejection.

The compositions and methods are further illustrated by the following non-limiting examples, which are not to be construed in any way as imposing limitations upon the scope thereof On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

The following experiments were conducted using methods and protocols well known to those skilled in the art. Details regarding the procedures used are found throughout the scientific literature and also for example in U.S. Pat. Nos. 5,981,471, 5,919,459, 6,346,510, and 6,413,513.

### EXAMPLES

### Example 1

### PAR Signalling Activity

Confluent HUVECs or HT29 colon carcinoma cells were loaded for 30-60 minutes with the fluorescent dye Fluo-4. Final concentration 4 uM Fluo-4, 0.02% pluronic acid in physiological buffer. Cells were then washed with assay buffer, (HBSS containing 1 mM CaCl₂, 1 mM MgSO₄, and 2.5 mM probenecid). Cells were stimulated with various doses of PAR-2 activating peptide, PAR-1 activating peptide or ATP. Fluorescence was monitored using a Wallac 1470 fluorescent plate reader. (See A1-ani et. al Journal of Pharmacology and Experimental Therapeutics 290:2, 753-760)

Calcium mobilization curves of the PAR-2 agonist SLIGKV (SEQ ID NO:25) compared with two truncated molecules LIGK (SEQ ID NO:1) and LIGKV (SEQ ID NO:2) are provided in FIG. 4A. Neither truncated molecule was able to induce calcium mobilization, in contrast with SLIGKV (SEQ ID NO:25), which demonstrates the typical spike of calcium release followed by degradation of signal. Similar studies were performed on alanine substituted SLIGKV (SEQ ID NO:25) peptides (FIGS. 4B and 4C). It was found that substitution of SLIGKV at S, L, I, or K abrogated or significantly diminished signaling activity, while two substituted peptides, SLIAKV (SEQ ID NO:31) and SLIGKA (SEQ ID NO:33) demonstrated robust signaling activity.

TABLE 1

| Peptide | SEQ ID NO: | Signal | Inhibit P2P |
|---|---|---|---|
| SLIGKV | SEQ ID NO: 25 | ++++ | NA |
| SLIGK | SEQ ID NO: 27 | ++ | NA |
| LIGKV | SEQ ID NO: 2 | − | + |
| LIGK | SEQ ID NO: 1 | − | ++++ |
| ALIGKV | SEQ ID NO: 28 | − | − |
| SAIGKV | SEQ ID NO: 29 | − | − |
| SLAGKV | SEQ ID NO: 30 | − | − |
| SLIAKV | SEQ ID NO: 31 | ++ | − |
| SLIGAV | SEQ ID NO: 32 | +/− | − |
| SLIGKA | SEQ ID NO: 33 | ++ | − |

### Example 2

### Identification and Testing of PAR-2 Antagonist

In order to assess the potential of peptides selected above to block PAR-2 signaling, cells were pretreated with potential antagonist peptides for a predetermined amount of time and were subsequently treated with P2AP. Methods and protocols used were the same as those described in Example 1. Two of the SLIGKV (SEQ ID NO:25) derived peptides demonstrated antagonist activity, LIGK (SEQ ID NO:1) and LIGKV (SEQ ID NO:2). FIG. 5 shows a representative dosing study where increasing concentrations of LIGK (SEQ ID NO:1) were used to block P2AP signaling. In this study, a concentration of 1 mM LIGK (SEQ ID NO:1) completely blocked the signaling of 100 uM SLIGKV (SEQ ID NO:25). In similar studies comparing the activity of LIGK (SEQ ID NO:1) with LIGKV (SEQ ID NO:2) it was found that the LIGK (SEQ ID NO:1) peptide is a more potent inhibitor of PAR-2 signaling (IC50<0.5 mM ), compared to LIGKV (SEQ ID NO:2) (FIG. 6).

### Example 3

### Activation Study for Assessing Inhibitory Activity of LIGK using A TP and SFLLRN

In order to demonstrate that LIGK (SEQ ID NO:1) is a specific inhibitor of PAR-2 signaling, activation studies were performed with ATP and the PAR-1 activation peptide, SFLLRN (SEQ ID NO:34), on cells that were pretreated with LIGK. Both of these molecules signal through G-protein coupled receptors, and PAR-1 is very highly homologous to PAR-2, to the degree that the PAR-1 agonist peptide can signal through PAR-2 at high concentrations. In both cases, the PAR-2 antagonist LIGK (SEQ ID NO:1) had no inhibitory effect on signaling (FIG. 7).

### Example 4

### In Vivo Analysis of LIGK Inhibitory Effect on PAR-2

C57b1/b mice had 5-25 μg of SLIGKV injected into their footpad, in the presence or absence of increasing amounts of various PAR-2 antagonists. One hour later, footpad (tarsus) thickness was measured to quantify inflammation (edema).

The inventors next assessed whether the LIGK peptide had in vivo PAR-2 antagonistic activity. This was studied using an edema model where vascular permeability was induced by the PAR-2 agonist peptide. In this model, the PAR-2 peptide induces severe edema as expected (FIG. 8). This vascular response was blocked by co-treatment with the PAR-2 antagonist LIGK (SEQ ID NO:1) (FIG. 9). Thus, LIGK (SEQ ID NO:1) functions in vivo to block PAR-2 signaling.

### Example 5

### Inhibitory Activity of LIGK in Lewis Lung Carcinoma Experimental Model

C57/B16 mice were injected i.v. with Lewis lung carcinoma. 3 days later, treatment of lung tumors was started with i.p. LIGK (SEQ ID NO:1) for 11 days.

In this model (FIG. 10), the PAR-2 antagonist LIGK was found to be a very potent inhibitor of metastatic tumor growth. At a dose of 4 mg/day tumor growth was inhibited by 75%. LIGK also demonstrated dose dependency across multiple independent studies, with an approximate IC50 of 2 mg/day (FIG. 11).

Similar experiments were performed in the LLC primary tumor model. In this model, treatment is initiated when tumor volume approaches 100 mm_{3.} Consistent with the metastasis model, LIGK proved to be a very potent inhibitor of LLC primary tumor (FIG. 12). At 1 mg/day, tumor growth was inhibited by 62%.

### Example 6

### Inhibitory Activity of LIGK in Matrigel Assay

C57/B16 mice were injected s.c. with Matrigel containing 0.5 μg FGF-2. Treatment was started at day 1 with LIGK administered s.c. for 6 days.

Matrigel plugs from animals treated with LIGK (SEQ ID NO:1) demonstrated a dose dependent inhibition of angiogenesis, based upon hemoglobin content in the plug (FIG. 13). At the highest dose of LIGK (SEQ ID NO:1), angiogenesis was inhibited by more than 80%. These data demonstrate that LIGK (SEQ ID NO:1) has potent antiangiogenic activity, and further suggest a mechanism by which LIGK (SEQ ID NO:1) could block tumor growth.

### Example 7

### Effect of LIGK (SEQ ID NO:1) on Arthritis in Mice

On day 0, Balb/c mice were injected IV with the 1-2 mg 1B11 monoclonal anti-collagen II antibody. On day 1, animals were injected i.p with 20 μg LPS, and treatment with PAR-2 antagonists (200 mg/kg/day i.p). for 7 days is initiated. After treatment was completed, disease is quantified by measuring the thickness (swelling) in both feet of the mouse. This was compared to untreated mice. (p<0.05 vs. vehicle control)

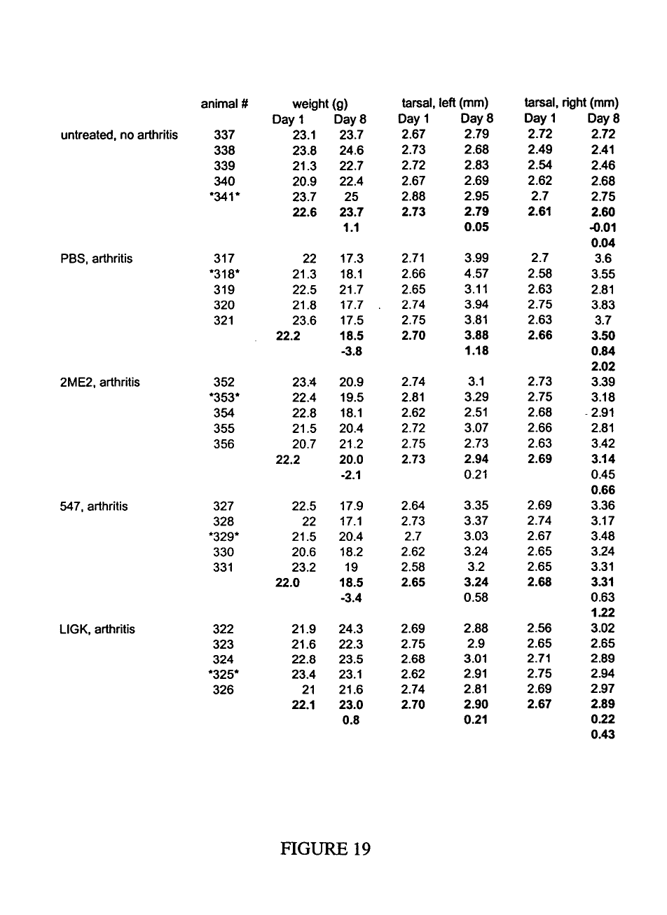

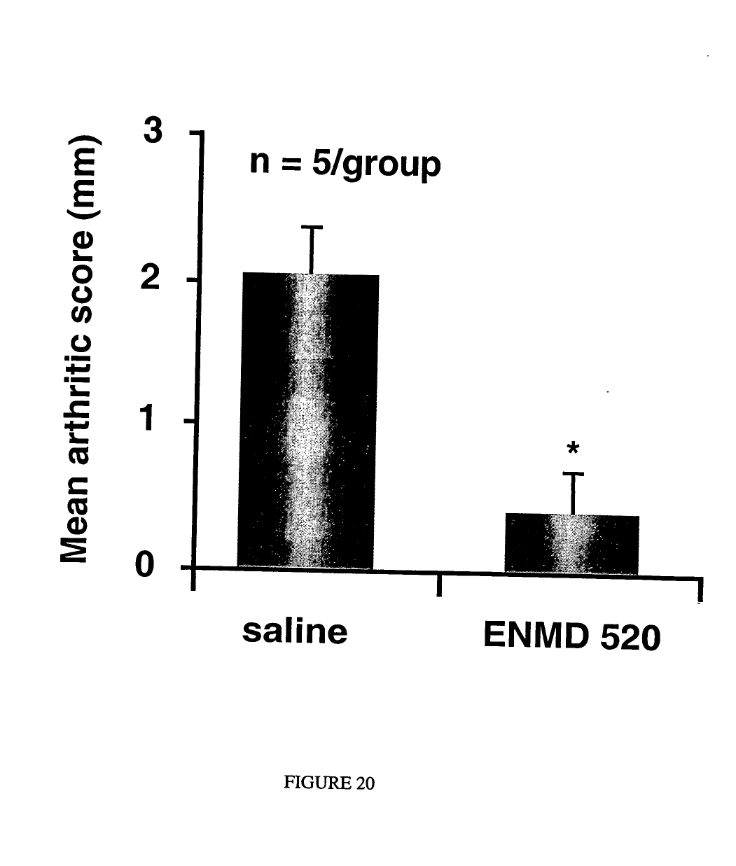

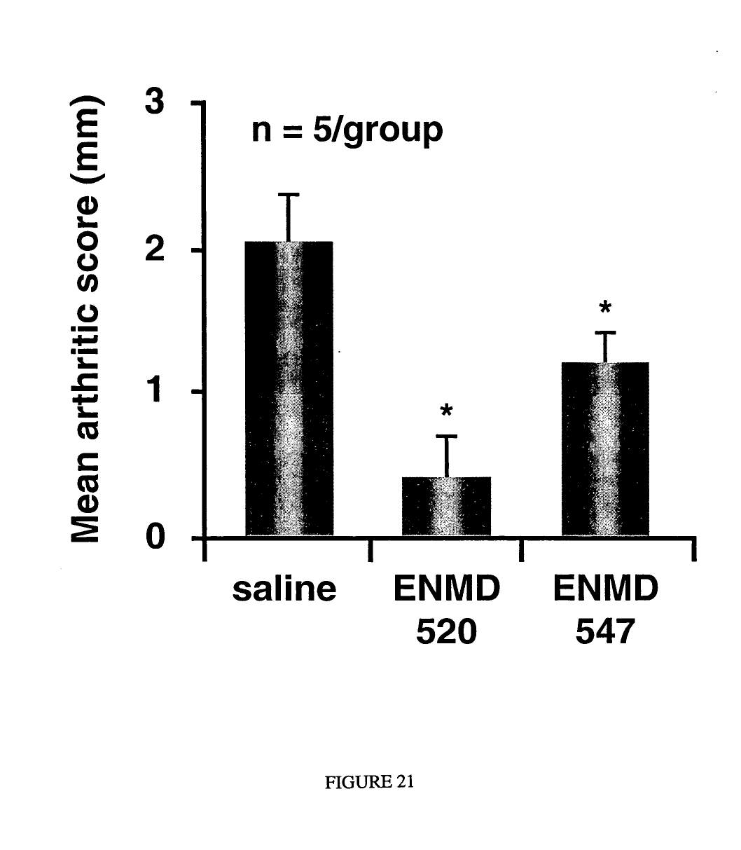

As shown in FIG. 19 both ENMD 547 and LIGK (SEQ ID NO:1) inhibited inflammation. FIG. 20 shows attenuation of arthritis in mice in the presence of LIGK (SEQ ID NO:1) (referred to as ENMD 520). FIG. 21 shows attenuation of arthritis in the presence of LIGK (SEQ ID NO:1) ENMD 520 and ENMD 547.

### Example 8

### Prevention of Arthrogen-CIA Induced Body Weight Loss in Mice

On day 0, Balb/c mice were injected IV with the 1-2 mg 1B 11 monoclonal anti-collagen II antibody. On day 1, animals were injected i.p with 20 μg LPS, and treatment with PAR-2 antagonists (200 mg/kg/day i.p). for 7 days is initiated. After treatment was completed, disease is quantified by measuring the thickness (swelling) in both feet of the mouse. This was compared to untreated mice This model results in significant weight loss associated with the administration of LPS. Treatment of these mice with LIGK abrogated this LPS induced weight loss.

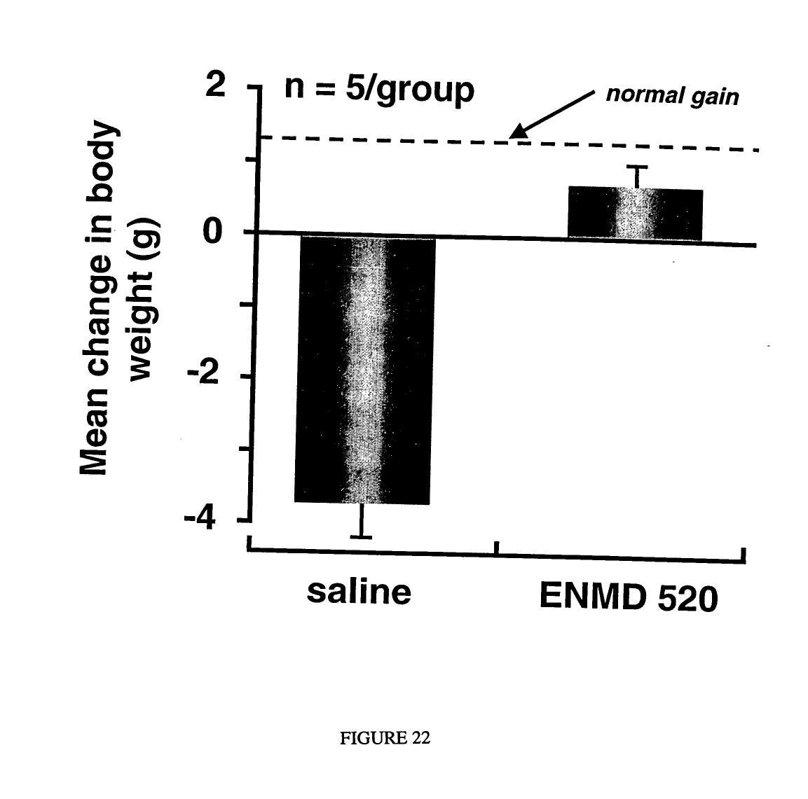

FIG. 22 shows prevention of weight loss in the presence of LIGK (SEQ ID NO:1), ENMD 520.

### Example 9

### In Vivo and In Vitro Activity of ENMD-547

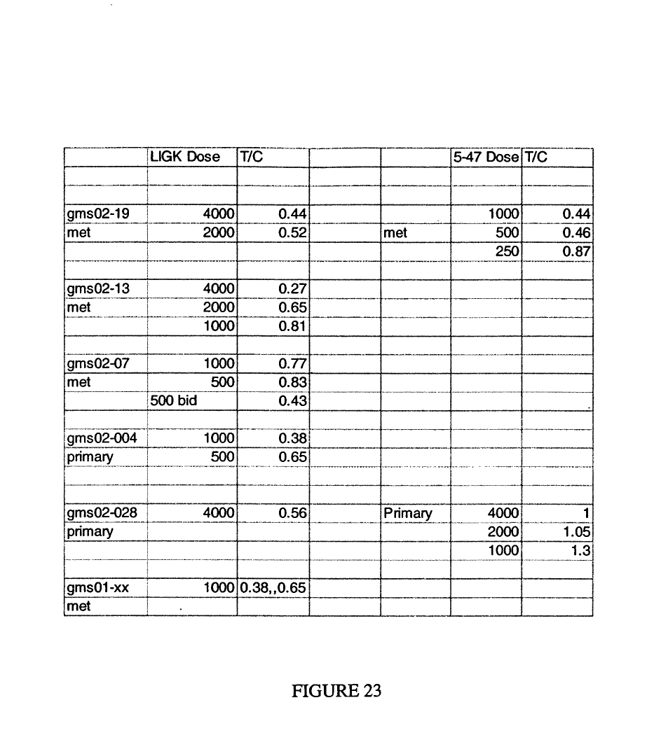

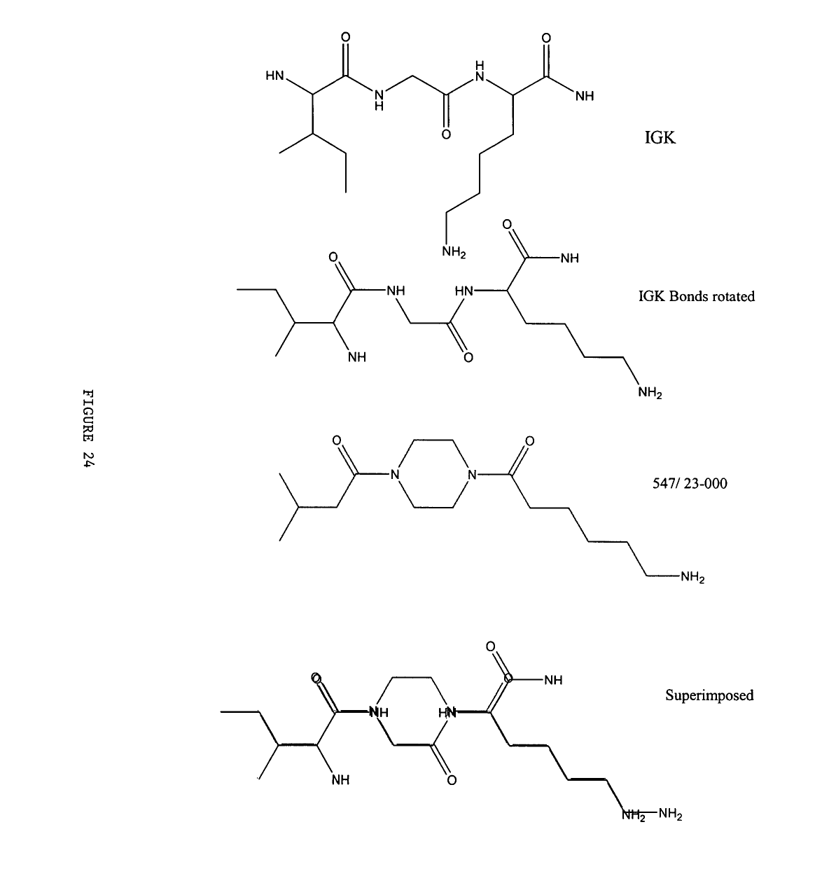

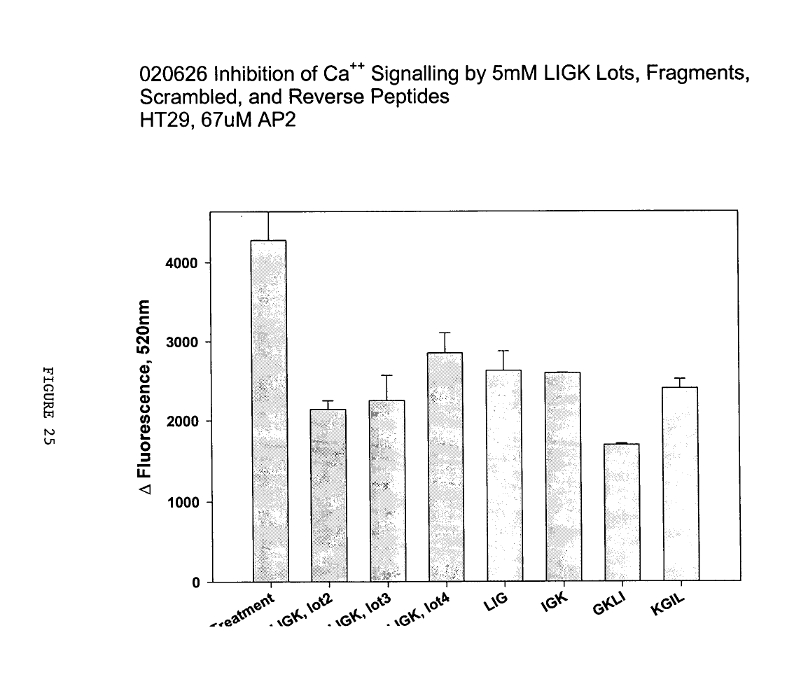

ENMD-547 was discovered to be an extremely potent inhibitor of PAR-2 signaling in vitro (FIG. 14). Like the LIGK peptide ENMD-547 has no inhibitory effects on signaling by ATP (FIG. 4c) or PAR-1 (not shown). Finally in metastatic tumor growth studies, ENMD-547 has potent antitumor activity, approximately five fold better than the parental LIGK molecule (FIG. 16). FIG. 23 provides antitumor data for LIGK (SEQ ID NO:1) and ENMD 547. Taken together, the identification of a second specific PAR-2 inhibitor with antitumor activity supports our contention that PAR-2 plays a vital role in the growth and development of tumors in vivo. In addition this molecule, due to its enhanced antitumor activity, may provide insight into the design and synthesis of other PAR-2 antagonist molecules.

1-20. (canceled)

21. A composition for inhibiting protein activated receptor activity associated with skin disorders comprising a protein, peptide, biomolecule or active fragment thereof.

22. The composition of claim 21 wherein the protein activated receptor comprises PAR-1, PAR-2, PAR-3 or PAR-4.

23. The composition of claim 21 wherein the protein, peptide, or biomolecule comprises LIGK (SEQ ID NO:1), LIGKV (SEQ ID NO:2), KGIL (SEQ ID NO:3), KGI (SEQ ID NO:4), AGI (SEQ ID NO:5), IGA (SEQ ID NO:6), KGA (SEQ ID NO:7), KGA (SEQ ID NO:8), KAI (SEQ ID NO:9), IAK (SEQ ID NO:10), RGI (SEQ ID. NO:11), IGR (SEQ ID NO:12), Dab-GI (SEQ ID NO:13 ), Dap-GI (SEQ ID NO:14), IG-Dab (SEQ ID NO:15 ), IG-Dap (SEQ ID NO:16), LIG-Dab (SEQ ID NO:17), Dab-GIL (SEQ ID NO:18), LIG-Dap (SEQ ID NO:19), Dap-GIL (SEQ ID NO:20), LIG-Orn (SEQ ID NO:21), Orn-GIL (SEQ ID: 22), Orn-GI (SEQ ID NO:23), IG-Orn (SEQ ID NO:24), ENMD 545, ENMD 547 and active fragments thereof.

24. The composition of claim 23, wherein the protein activated receptor comprises PAR-2.

25. The composition of claim 21, wherein the protein activated receptor comprises PAR-2 and wherein the wherein the protein, peptide, or biomolecule comprises LIGK (SEQ ID NO:1).

26. The composition of claim 21, wherein the protein activated receptor comprises PAR-2 and wherein the wherein the protein, peptide, or biomolecule comprises ENMD 547.

27. The composition of claim 21, further comprising a pharmaceutically acceptable carrier.

28. The composition of claim 21, wherein the skin disorder comprises psoriasis, telangiectasia psoriasis, scleroderma, wound healing, delayed wound healing, hand foot and mouth disease, hair growth, rheumatoid arthritis, rosacea, skin warts, scars, Bechet's disease and inflammatory disease.

29. The composition of claim 21, wherein the skin disorder comprises eczema, dermatitis, seborrheic dermatitis, contact dermatitis, atopic dermatitis, nummular dermatitis, chronic dermatitis, Lichen Simplex Chronicus, stasis dermatitis, and generalized exfoliative dermatitis.

30. The composition of claim 21, wherein the skin disorder comprises psoriasis.

31. A method for inhibiting protein activated receptor activity associated with skin disorders in a subject comprising administering to said subject protein, peptide, biomolecule or active fragment thereof.

32. The method of claim 31, wherein the protein activated receptor comprises PAR-1, PAR-2, PAR-3 or PAR-4.

33. The method of claim 31, wherein the protein, peptide, or biomolecule comprises LIGK (SEQ ID NO:1), LIGKV (SEQ ID NO:2), KGIL (SEQ ID NO:3), KGI (SEQ ID NO:4), AGI (SEQ ID NO:5), IGA (SEQ ID NO:6), KGA (SEQ ID NO:7), KGA (SEQ ID NO:8), KAI (SEQ ID NO:9), IAK (SEQ ID NO:10), RGI (SEQ ID. NO:11), IGR (SEQ ID NO:12), Dab-GI (SEQ ID NO:13 ), Dap-GI (SEQ ID NO:14), IG-Dab (SEQ ID NO:15 ), IG-Dap (SEQ ID NO:16), LIG-Dab (SEQ ID NO:17), Dab-GIL (SEQ ID NO:18), LIG-Dap (SEQ ID NO:19), Dap-GIL (SEQ ID NO:20), LIG-Orn (SEQ ID NO:21), Orn-GIL (SEQ ID: 22), Orn-GI (SEQ ID NO:23), IG-Orn (SEQ ID NO:24), ENMD 545, ENMD 547 and active fragments thereof.

34. The method of claim 31, wherein the protein activated receptor comprises PAR-2.

35. The method of claim 31, wherein the protein activated receptor comprises PAR-2 and wherein the wherein the protein, peptide, or biomolecule comprises LIGK (SEQ ID NO:1).

36. The method of claim 31, wherein the protein activated receptor comprises PAR-2 and wherein the wherein the protein, peptide, or biomolecule comprises ENMD 547.

37. The method of claim 31, further comprising a pharmaceutically acceptable carrier.

38. The composition of claim 31, wherein the skin disorder comprises psoriasis, telangiectasia psoriasis, scleroderma, wound healing, delayed wound healing, hand foot and mouth disease, hair growth, rheumatoid arthritis, rosacea, skin warts, scars, and inflammatory disease.

39. The composition of claim 31, wherein the skin disorder comprises eczema, dermatitis, seborrheic dermatitis, contact dermatitis, atopic dermatitis, nummular dermatitis, chronic dermatitis, Lichen Simplex Chronicus, stasis dermatitis, and generalized exfoliative dermatitis.

40. The composition of claim 31, wherein the skin disorder comprises psoriasis.


SEQUENCE LISTING (34 sequences)

SEQ ID NO: 1 - protein, 4 aa, Artificial Sequence
  Synthetic
LIGK

SEQ ID NO: 2 - protein, 5 aa, Artificial Sequence
  Synthetic
LIGKV

SEQ ID NO: 3 - protein, 4 aa, Artificial Sequence
  Synthetic
KGIL

SEQ ID NO: 4 - protein, 3 aa, Artificial Sequence
  Synthetic
KGI

SEQ ID NO: 5 - protein, 3 aa, Artificial Sequence
  Synthetic
AGI

SEQ ID NO: 6 - protein, 3 aa, Artificial Sequence
  Synthetic
IGA

SEQ ID NO: 7 - protein, 3 aa, Artificial Sequence
  Synthetic
KGA

SEQ ID NO: 8 - protein, 3 aa, Artificial Sequence
  Synthetic
KGA

SEQ ID NO: 9 - protein, 3 aa, Artificial Sequence
  Synthetic
KAI

SEQ ID NO: 10 - protein, 3 aa, Artificial Sequence
  Synthetic
IAK

SEQ ID NO: 11 - protein, 3 aa, Artificial Sequence
  Synthetic
RGI

SEQ ID NO: 12 - protein, 3 aa, Artificial Sequence
  Synthetic
IGR

SEQ ID NO: 13 - protein, 3 aa, Artificial Sequence
  Synthetic
XGI

SEQ ID NO: 14 - protein, 3 aa, Artificial Sequence
  Synthetic
XGI

SEQ ID NO: 15 - protein, 3 aa, Artificial Sequence
  Synthetic
IGX

SEQ ID NO: 16 - protein, 3 aa, Artificial Sequence
  Synthetic
IGX

SEQ ID NO: 17 - protein, 4 aa, Artificial Sequence
  Synthetic
LIGX

SEQ ID NO: 18 - protein, 4 aa, Artificial Sequence
  Synthetic
XGIL

SEQ ID NO: 19 - protein, 4 aa, Artificial Sequence
  Synthetic
LIGX

SEQ ID NO: 20 - protein, 4 aa, Artificial Sequence
  Synthetic
XGIL

SEQ ID NO: 21 - protein, 4 aa, Artificial Sequence
  Synthetic
LIGX

SEQ ID NO: 22 - protein, 4 aa, Artificial Sequence
  Synthetic
XGIL

SEQ ID NO: 23 - protein, 3 aa, Artificial Sequence
  Synthetic
XGI

SEQ ID NO: 24 - protein, 3 aa, Artificial Sequence
  Synthetic
IGX

SEQ ID NO: 25 - protein, 6 aa, Homo sapiens
SLIGKV

SEQ ID NO: 26 - protein, 6 aa, Murinae gen. sp.
SLIGRL

SEQ ID NO: 27 - protein, 5 aa, Artificial Sequence
  Synthetic
SLIGK

SEQ ID NO: 28 - protein, 6 aa, Artificial Sequence
  Synthetic
ALIGKV

SEQ ID NO: 29 - protein, 6 aa, Artificial Sequence
  Synthetic
SAIGKV

SEQ ID NO: 30 - protein, 6 aa, Artificial Sequence
  Synthetic
SLAGKV

SEQ ID NO: 31 - protein, 6 aa, Artificial Sequence
  Synthetic
SLIAKV

SEQ ID NO: 32 - protein, 6 aa, Artificial Sequence
  Synthetic
SLIGAV

SEQ ID NO: 33 - protein, 6 aa, Artificial Sequence
  Synthetic
SLIGKA

SEQ ID NO: 34 - protein, 6 aa, Artificial Sequence
  Synthetic
SFLLRN